(19) United States
(12) Patent Application Publication
(10) Pub. No.: US 2007/0009986 A1
(43) Pub. Date: Jan. 11, 2007

(54) Compositions for modulating immune cell activity and methods for detection thereof

(76) Inventors: Mark C. Poznansky, Charlestown, MA (US); Diana Brainard, Boston, MA (US)

Correspondence Address: EDWARDS & ANGELL, LLP, P.O. BOX 55874, BOSTON, MA, 02205, US

(21) Appl. No.: 10/543,850

(22) PCT Filed: Jan. 30, 2004
(86) PCT No.: PCT/US04/02592

§ 371 Date: Sep. 1, 2006

Related U.S. Application Data
(60) Provisional application No. 60/443,637, filed on Jan. 30, 2003.
(60) Provisional application No. 60/446,458, filed on Feb. 10, 2003.

(51) Int. Cl. C12Q 1/70; C12Q 1/68; C12P 21/06

(52) U.S. Cl. 435/69.1; 435/6; 435/5

(57) ABSTRACT

The invention relates to a new method for measuring cytotoxic activity of immune cells, and to methods and products for treating abnormal immune responses.

### CROSS-REFERENCE TO RELATED APPLICATIONS

This application claims priority to U.S. Application Ser. No. 60/443,647, filed Jan. 30, 2003 and U.S. Application Ser. No. 60/446,458, filed Feb. 10, 2003.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the numbered paragraphs, or in the text itself; and, each of these documents or references (“herein-cited references”), as well as each document or reference cited in each of the herein-cited references (including any manufacturer's specifications, instructions, etc.), is hereby expressly incorporated herein by reference.

### STATEMENT OF RIGHTS TO INVENTIONS MADE UNDER FEDERALLY SPONSORED RESEARCH

Aspects of the invention may have been made using funding from the National Institutes of Health, Grant No. R01 A149757-02. Accordingly, the Government may have rights in the invention.

### FIELD OF THE INVENTION

The invention relates to a new method for measuring cytotoxic activity of immune cells, and to methods and products for treating abnormal immune responses.

### BACKGROUND OF THE INVENTION

Cells with cytotoxic activity contribute greatly to immune responses. In the treatment of certain disorders (e.g., cancer, infection), an enhanced immune response is beneficial and therefore, could be aided by increases in cytotoxic activity. For example, treatment of HIV infection could benefit from the ability to improve cytotoxic effects. Cytotoxic T lymphocytes (CTLs) have been implicated as essential but not sufficient to provide a robust immune response directed to HIV infection. (Addo et al. 2003 J. Virol. 77:2081.) HIV infection is thought to evade immune surveillance for various reasons including loss of CD4+ T cells, viral mutational escape of HIV virions, and direct effects of HIV proteins (such as nef). (See review by Greene et al. 2002 Nature Med. 8:673.) Improving CTL cytotoxic activity against HIV virons would potentially enhance the overall immune response against HIV infection.

Conversely, treatment of autoimmune diseases could potentially be aided by the ability to downregulate T cell activity. Autoimmune diseases are characterized by powerful immune responses to self-antigens that cause unwanted effects in a host. The ability to decrease CTL cytotoxic activity could decrease cellular damage that is associated with autoimmunity.

Thus, modulation of immune activity through regulation of cytotoxic effects could be important in treatment of various conditions. Accordingly, there is a need for methods and compositions that effectively modulate immune activity. In particular, there is a need for methods and compositions that effectively modulate CTL cytotoxic activity.

In order to most effectively identify such compositions, screening assays should be conducted in a physiological setting. Screening for cytotoxic activity currently involves chromium release assays, which are used to measure the ability of agents to induce cells including CTLs to lyse (rupture) specific target cells. This assay generally involves placing CTLs and their chromium-labeled targets into a round-bottom well of a 96-well tissue culture plate. The cells are then incubated for a period of time, during which they settle in close proximity to each other at the bottom of the round well. After four hours, supernatant is harvested, and its chromium content is measured. When placed in a round-bottomed culture plate, the CTLs are positioned next to the target cells. However, by forcing an association between CTLs and target cells, total CTL activity is not properly assessed because the ability of CTLs to actively migrate to the target cell is not considered. Improved methods that provide physiological conditions for screening would be desirable in the detection of compositions that modulate CTL cytotoxic activity.

### SUMMARY OF THE INVENTION

It has now been discovered that active migration of CTLs is required for effective destruction of target cells. Therefore, the cytotoxic effect of CTLs can vary when the distance between a CTL and a target cell is varied. Accordingly, migration of CTLs towards target cells must be considered and accounted for in order to quantitate CTL activity in vitro.

Methods of the invention provide a complete measure of cytotoxic effects in vivo because the critical role of cell migration in cytotoxicity is now evaluated.

At least three improvements contribute to the methods of the invention. First, a flat bottom is used in culture so that the cells do not pellet together at the bottom, but rather, are physiologically distributed. Second, the total number of cells is generally held constant. Third, it has now been shown that an inverse relationship exists between the effector-to-target cell distance and killing efficacy. Accordingly, the distance that effector cells must travel in order to lyse target cells is considered in measures of cytotoxicity. The average distance between an effector cell and a target cell can be calculated using a mathematical model that takes into account effector/target ratio, as well as the total number of cells and the size of the well. However, the invention is not limited to the use of such a model; standard curves can be generated without it, as will be described in greater detail herein.

Therefore, for any given effector cell, a set of standard curves can be determined which demonstrate the effect of altering the distance that the cytotoxic cell must travel to reach a target cell at varying effector/target ratios and constant densities. These relationships can be used to compare efficacy of different effector cells and also to study the effect of proteins expressed by a particular target cell, such as a virus-infected cell or cancer cell, which may alter the migratory pattern of cytotoxic cells, thereby altering their cytotoxic effect.

Accordingly, in one aspect, the invention provides a method for measuring cytotoxic activity of immune cells having cytotoxic activity (i.e., “cytotoxic cells” or “cytotoxic immune cells”). The method comprises placing at least one effector cell and at least one target cell in a flat bottom chamber, incubating the cells for a time sufficient to allow killing of the at least one target cell by the at least one effector cell, and determining a proportion of target cells killed, wherein the proportion of target cells killed is measured using a non-fluorescent assay. In one embodiment, the immune cell having cytotoxic activity is a cytotoxic T lymphocyte, but it is not so limited. In this and other related aspects of the invention, the immune cell may be a natural killer (NK) cell, a neutrophil, a cytotoxic CD4+ T lymphocyte, a macrophage, or a dendritic cell.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

In one embodiment, the non-fluorescent assay comprises release of radioactivity. In one embodiment, the radioactivity released is radiolabeled chromium (e.g., ⁵¹Cr release).

In one embodiment, the at least one effector cell and the at least one target cell are present in a pre-defined ratio. The ratio is not intended to limit the invention. It may range from 1000:1 to 1:1. In other embodiments, the predefined ratio is 750:1, 500:1, 250:1, 100:1, 50:1, 10:1 or 5:1.

In this and other aspects of the invention, the absolute number of cells in the well may be constant. The number of cells per well is not intended to limit the invention. The number of cells per well can range from 10,000 to 200,000, but it is not so limited. In specific embodiments, the number of cells per well is at least 10,000, at least 20,000, at least 25,000, at least 50,000, at least 75,000, at lest 100,000, at least 125,000, at least 150,000, at least 175,000, and at least 200,000 cells per well.

In one embodiment, the method further comprises comparing results of the assay to a standard curve. The standard curve may be generated using a control population of target cells. Alternatively, the extent of cytotoxicity may be determined as a proportion of control target cell lysis.

In another embodiment, the method further comprises determining distance between the effector cells and target cells.

In still another aspect, the invention provides a method for measuring activity of immune cells having cytotoxic activity. The method comprises placing at least one effector cell and at least one target cell in a flat bottom chamber, incubating the cells for a time sufficient to allow lysing of the at least one target cell by the at least one effector cell, and determining a proportion of target cells lysed, wherein the proportion of target cells lysed is measured using a flow cytometer or a radioactivity counter. In an important embodiment, the immune cell having cytotoxic activity is a cytotoxic T lymphocyte.

In this and other aspects of the invention, either the proportion or absolute number of target cellss lysed can be determined. However, given that the number of targets may differ between wells, it may be generally more appropriate to determine proportion rather than absolute number.

In one embodiment, the radioactivity counter is used to measure release of radioactivity, such as release of radiolabeled chromium. In another embodiment, the flow cytometer is used to measure propidium iodide uptake, 7-AAD uptake, uptake of fluorogenic caspase substrates such as but not limited to PhiPhiLux or fluorochrome-conjugated activated caspase antibodies.

In one embodiment, the at least one effector cell and the at least one target cell are present in a pre-defined ratio. The ratio is not intended to limit the invention. It may range from 1000:1 to 1:1. In other embodiments, the predefined ratio is 750:1, 500:1, 250:1, 100:1, 50:1, 10:1 or 5:1.

In one embodiment, the method further comprises comparing results of the assay to a standard curve.

In another aspect, the invention provides a method for measuring activity of immune cells having cytotoxic activity comprising placing at least one effector cell and at least one target cell in a flat bottom chamber, incubating the cells for a time sufficient to allow lysing of the at least one target cell by the at least one effector cell, determining a proportion of target cells lysed, and comparing the proportion of target cells lysed to a standard curve.

In another method, the proportion of target cells killed is measured by fluorescence or radioactivity release. In another embodiment, the proportion of target cells lysed is measured using a flow cytometer or a radioactivity counter. In a related embodiment, the radioactivity counter is used to measure radioactive chromium release. In another related embodiment, the flow cytometer is used to measure propidium iodide uptake, or other fluorescent markers as described herein.

In one embodiment, the at least one effector cell and the at least one target cell are present in a pre-defined ratio. The ratio is not intended to limit the invention. It may range from 1000:1 to 1:1. In other embodiments, the predefined ratio is 750:1, 500:1, 250:1, 100:1, 50:1, 10:1 or 5:1.

In yet another aspect, the invention provides a method for measuring activity of immune cells having cytotoxic activity comprising placing at least one effector cell and at least one target cell in a flat bottom chamber, determining a migration rate of the at least one effector cell towards the at least one target cell, determining a proportion of target cells lysed, and comparing the migration rate and the proportion of cells lysed with a standard curve.

It has been further been discovered, by carrying out methods of the invention, that the HIV gp120 protein causes fugetaxis of immune cells having cytotoxic activity such as CTLs (i.e., migration of CTLs away from the location of the gp120), thereby explaining at least in part the inability of the immune system to eradicate an HIV infection. This finding has led to the observation that agents which inhibit gp120 mediated fugetaxis can be used therapeutically to treat or prevent a condition that would benefit from increased immune cell involvement such as but not limited to an HIV infection. It has further lead to the observation that gp120 itself can be used therapeutically in subjects undergoing an abnormal immune response or in subjects that would benefit from a decreased immune cell involvement. An example is an undesired infiltration of immune cells such as T lymphocytes into a site within a subject (e.g., during RSV infection in newborns).

Accordingly, in one aspect, the invention provides a method for inhibiting an abnormal immune response comprising administering to a subject in need thereof a gp120 molecule or functional equivalent thereof in an amount effective to inhibit an abnormal immune response.

In one embodiment, the abnormal immune response includes undesired infiltration of T lymphocytes. In another embodiment, the soluble gp120 inhibits the undesired infiltration of T lymphocytes to a site within a subject.

In one embodiment, the abnormal immune response is selected from the group consisting of autoimmune disease, immune hypersensitivity, allergy, asthma, graft-versus-host disease (GVHD), and inflammation. In another embodiment, the abnormal immune response is reduced to a normal level.

In another aspect, the invention provides a method for enhancing migration of antigen-specific immune cells towards an antigen-expressing target comprising administering to a subject in need thereof an agent that inhibits gp120-mediated fugetaxis in an amount effective to enhance migration of antigen-specific immune cells towards an antigen-expressing target.

In one embodiment, the antigen-specific immune cells are T lymphocytes which in turn may be cytotoxic T lymphocytes. The antigen-specific immune cells may also be natural killer (NK) cells, neutrophils, macrophages, cytotoxic CD8+ T lymphocytes, cytotoxic CD4+ T lymphocytes, or dendritic cells. In another embodiment, the antigen-expressing target is an HIV antigen-expressing target, such as a cell free HIV virus or a cell-associated HIV virus.

In one embodiment, the agent is selected from the group consisting of an anti-chemokine receptor antibody or a fragment thereof (such as anti-CXCR-4 antibody or a fragment thereof or anti-CXCR-5 antibody or a fragment thereof), a G-alpha-I inhibitor (such as a pertussis toxin or a functional equivalent thereof), a kinase inhibitor (such as a phosphatidylinositol 3-kinase (PI3-K) inhibitor, e.g., wortmannin, or a tyrosine kinase inhibitor, e.g., genistein or herbimycin), and a cAMP agonist (such as a cyclic nucleotide, e.g., 8-Br-cAMP or a functional equivalent thereof). In another embodiment, the agent is administered systemically or in a sustained release vehicle.

In one embodiment, where the method is directed to a subject having or at risk of developing an HIV infection, the method further comprises administering an anti-HIV agent to the subject. In one embodiment, the subject has an HIV infection. In another embodiment, the subject is at risk of developing an HIV infection. In yet another embodiment, the subject has been exposed to HIV.

These and other embodiments of the invention will be described in greater detail herein.

### BRIEF DESCRIPTION OF THE FIGURES

FIG. 1 depicts transmigration responses of a representative HIV-specific CTL clone (161JD27) using recombinant HIV-1_{IIIB} gp120 at concentrations of 20 ng/ml and 200 ng/ml.

FIG. 2 shows that Pertussis toxin and anti-CXCR4 antibodies inhibit active movement of T-cells towards and away from X4 gp120 when incubated with the G αᵢ, inhibitor, permssis toxin (q), or anti-CXCR-4 antibodies (0), prior to their addition to the transmigration assay.

FIG. 3 depicts migration of CD8⁺ T-cells in response to intact and X4 HIV-1 gp120 containing variable loop deletions.

FIG. 4 depicts modifications to the standard ⁵¹Cr release assay demonstrate that CTL migration influences killing efficacy. (A) CTL killing in the standard ⁵¹Cr assay in round bottom 96 well plates was compared to experiments done in a flat bottom plate. (B) The standard assay in the flat bottom well plate was performed in parallel with a modified ⁵¹Cr assay where the total number of cells was kept constant at 110,000 per well and only the E:T ratio was changed. (C) Correlation of the mathematical model to experimental data.

FIG. 5 depicts effects of X4 gp120 expression by target cells on CTL lysis.

FIG. 6 shows that X4 HIV-1 gp120 abrogates T-cell infiltration into a site of antigen challenge in vivo. C57/BL6 (FIG. 6A) and OT-1 Mice (FIG. 6B) were immunized with Ova subcutaneously, later challenged with intraperitoneal (IP) Ova (time 0) and 24 hours after IP Ova injection, one of several forms of recombinant X4 HIV-1 gp120: HIV-1_{IIIB} gp120 (/), HIV-1_{IIIB} gp120ΔV1 V2 (1, [A]), HIV-1 HIV-1_{IIIB} gp120ΔV1 V2V3 (X) was administered.

FIG. 7 depicts a series of nucleotide and amino acid sequences of gp120 from GenBank.

It is to be understood that the Figures are not required for enablement of the invention.

### DETAILED DESCRIPTION OF THE INVENTION

The invention provides methods for conducting novel assays to measure cytotoxic activity. Methods of the invention allow for precise and physiological measurement of an immune cell's ability to lyse specific target cells (i.e., cytotoxic activity). Using methods of the invention, test agents, such as proteins or other small molecules, can be evaluated to determine their positive or negative effect on cell movement, as well as their overall positive or negative effect on cytotoxicity.

At least three improvements contribute to the methods of the invention. First, a flat bottom is used in culture so that the cells do not pellet together at the bottom, but rather, are physiologically distributed. Second, the total number of cells is generally held constant. Third, it has now been shown that an inverse relationship exists between the effector-to-target cell distance and killing efficacy. Accordingly, the distance that effector cells must travel in order to lyse target cells is considered in measures of cytotoxicity. The average distance between an effector cell and a target cell can be calculated using a mathematical model that takes into account effector/target ratio, as well as the total number of cells and the size of the well. However, the invention is not limited to the use of such a model; standard curves can be generated without it, as will be described in greater detail herein.

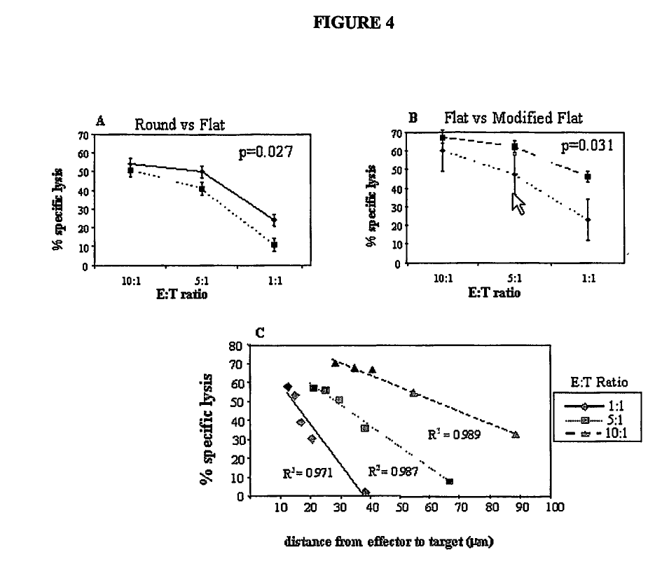

The distribution of cells in a round bottom (top panel) versus a flat bottom (bottom panel) chamber is shown in FIG. 4A. There is clustering of cells in the round bottom chambers regardless of cell density as compared to the flat bottom chambers. The use of flat bottom chambers versus round bottom chambers increased the amount of specific lysis. FIG. 4B indicates that more killing was observed when flat bottom wells were used relative to round bottom wells, regardless of the type of effector clone used or effector:target ratio.

Furthermore, it has now been determined that changes in the effector:target ratio and changes in the total number of cells per chamber impact cytotoxic activity. Thus, in preferred embodiments, the total number of cells is held constant in all wells.

It has also been determined that an inverse correlation exists between the effector-to-target cell distance and killing efficacy (cytotoxicity). Thus, in preferred embodiments, the average distance that an effector cell travels to reach a target cell is calculated and considered in determining cytotoxicity. Calculation of the distance is dependent upon factors including the total number of cells in the chamber, the ratio of effector:target cells, and the size of the chamber (e.g., volume of the chamber and/or surface area of the flat bottom).

The distance between effector and target cells can be determined using a mathematical model, however, the invention is not dependent upon the use of a model, as it is possible to compare different wells simply based on a plot of specific cell lysis versus effector:target ratio. Use of a mathematical model allows for the data to be plotted in a linear format, as shown in FIG. 4C; however, as stated above, this is not necessary in order to practice the invention. The mathematical model illustrates that the distance between effector and target cells increases with increasing effector-to-target ratios, regardless of the total number of cells per well. This is so, provided that the total number of cells in the various wells is kept constant even if the ratio changes.

Methods of the invention enable screening of test agents (e.g., such as proteins or other small molecules) for the ability to modulate cytotoxic activity by influencing migration of immune cells having cytotoxic activity. Compounds that stimulate chemotaxis (i.e., movement towards an agent) or fugetaxis (i.e., movement away from an agent) can be identified using methods of the invention.

Methods of the invention enable screening of test agents for the ability to overcome chemotactic or fugetactic forces acting on a subject. For example, as shown herein, the HIV-specific gp120 protein causes fugetaxis of T lymphocytes. Using methods of the invention, test agents can be screened in the presence of gp120 and T lymphocytes for the ability to overcome the fugetactic influence of gp120. Compounds so identified can potentially be used therapeutically in subjects having or at risk of developing (e.g., those exposed to) HIV infection.

Cytotoxic activity can be measured using techniques known in the art, as well as those described herein. The non-fluorescent assay may be radioactivity release. A radioactivity release assay is one that uses target cells that are first loaded with radioactivity such as for example radioactive chromium (e.g., ⁵¹Cr). Once target cells are incubated with and killed by the effector cells, their radioactive contents are released into the medium and this released radioactivity can then be detected using a radioactivity counter. The invention is not limited to the radioactive compound used, and those of ordinary skill will be able to routinely modify the assay for other radioactivity types.

In other aspects of the invention, the assay is a fluorometric assay that detects fluorescent signal or lack thereof as an indicator of cell lysis during incubation. As an example, a target cell not initially loaded with a fluorescent marker is incubated with an effector cell. Once the incubation is complete, a fluorescent marker is added to the culture and allowed to enter any dead target cells. Dead cells generally have a perforated cell membrane and thus solutes including fluorescent markers can be easily taken up by these cells. One such marker is propidium iodide which enters the cell and binds to the DNA. The cells can then be washed in order to remove any fluorophore that is not within the dead cells, and then analyzed using either a flow cytometer or a fluorescence microplate reader. Other fluorescent markers include 7-AAD, fluorogenic caspase substrates (e.g., PhiPhiLux or fluorochrome-conjugated activated caspase antibodies). 7-AAD does not stain viable cells generally, although it stains dying cells to a low level, and dead cells to a high level. In some embodiments, the cells are analyzed with a flow cytometer. The use of flow cytometry allows one to distinguish between dead effector cells and dead target cells by using a second marker that is either present on the effector cell but not on the target cell, or vice versa.

The invention involves in some aspects immune cells with cytotoxic activity. A immune cell as used herein is a cell of hematopoietic origin that is involved in the recognition of antigens. Immune cells include antigen presenting cells (APCs), such as dendritic cells or macrophages, B cells, T cells, neutrophils, natural killer (NK) cells, etc. “Mature T cells” as used herein include T cells of a CD4^{lo}CD8^{hi}CD69⁺TCR⁺, CD4^{hi}CD8^{lo}CD69⁺TCR⁺, CD4⁺CD45⁺RA⁺, CD4⁺CD3⁺RO⁺, and/or CD8⁺CD3⁺RO⁺ phenotype. An immune cell with cytotoxic activity is an immune cell that is capable of killing another cell. In some embodiments, the cell kills its target directly; in other embodiments, it kills its target indirectly.

As used therein a “T cell” and a “T lymphocyte” are used interchangeably and assume their ordinary meaning. A cytotoxic T cell is generally a T cell having cytotoxic activity such as a CD8+ T cell. It has recently been reported that a subset of CD4+ lymphocytes are also capable of cytotoxic activity. Accordingly, the cytotoxic cells of the invention include CD4+ cytotoxic T lymphocytes as well.

The cytotoxicity assays provided by the invention generally employ two cell types: effector cells and target cells. As used herein, an “effector cell” is any cell having, or believed to have, cytotoxic activity. “Cytotoxic” refers to the toxic effect on a target cell that is produced by an effector cell. The toxic effect causes the target cell to lyse (i.e., rupture). Cytotoxicity is also referred to herein as “cell killing,” and the strength of the cytotoxic effect can be referred to as “killing efficacy.”

An effector cell is generally an immune cell with cytotoxic activity. Effector cells are cells that cause the killing in a cytotoxicity assay. In some instances it is also an antigen-specific immune cell. They may be any cell that is capable of killing other cells, but usually are immune cells such as cytotoxic T cells. Target cells are the cells that will be recognized and killed by the effector cells in a cytotoxicity assay. The target cell may be any cell that is recognized by an immune cell with cytotoxic activity. Examples of suitable target cells include virus-infected cells and tumor cells. The effector cells are generally non-adherent as they must be capable of migrating within the chamber to the site of a target cell. The target cell may be adherent or non-adherent. In some embodiments, the target cell is non-adherent.

In carrying out methods of the invention, cells are incubated for a time sufficient to allow the effector cells to kill the target cells. This time is determined on a case by case basis and one of ordinary skill in the art is familiar with suitable time ranges. The time can be as little as 30 minutes to 12 hours or more, including every time therebetween. In preferred embodiments, the time ranges from 2 to 4 hours. The incubation can be carried out at 4, 25 or 37 degrees Celsius, or at room temperature. In preferred embodiments, the incubation is carried out at 37 degrees Celsius. Preferably, the medium in which the cells are placed during the incubation contains the nutrients required to keep the cells otherwise healthy and viable. Accordingly, any cell death is attributable to the action of the effector cell on the target cell.

The proportion of cells killed can be determined by comparison with an a priori determination of the signal to be generated by the death of a given number of cells. For example, this can be done by running controls that involve lysis of a defined number of target cells and then measuring either the amount of chromium in the medium or the amount of fluorescent signal in the well after washing (particularly if using a microplate fluorescence reader). If a flow cytometer is used, it should be possible to directly count the number of fluorescently labeled cells.

Some embodiments of the invention require that multiple and potentially parallel cytotoxic assays be performed. These assays may all include the same effector to target ratio, or they may include differing ratios. In some important embodiments, the effector to target cell ratio is a pre-defined ratio. The ratio is not intended to limit the invention. It may range from 1000:1 to 1:1. In other embodiments, the predefined ratio is 750:1, 500:1, 250:1, 100:1, 50:1, 10:1 or 5:1. These ratios may be used both in the actual assay and when deriving a standard curve for a particular source of effector and/or target cells. The ratio of cells will depend upon the nature and activity of the effector cell, the target cell, or both. Those of ordinary skill in the art will be familiar with determining a suitable ratio based on the characteristics of the cells involved and the volume and/or area of the chamber.

The total number of cells per well will vary depending upon the volume of the chamber, and particularly the surface area of flat bottom of the chamber. For example, the small the chamber, the fewer the cells that will be added, generally. The number of cells per well within a given assay may be constant. A constant cell number means that in a given assay with multiple wells wherein wells contain differing ratios of effector to target cells, the absolute number of cells (i.e., the combined total of effector and target cells) is the same regardless of the ratio of cells. For example, in a given assay, there may be at least three wells with differing ratios (e.g., 100:1, 50:1 and 10:1). The total number of cells in each well is the same (e.g., 100,000 cells per well). This means that the total number of each cell type will vary between wells. If the ratio is 100:1, then there will be approximately 99010 effector cells and 990 target cells (for a total of 100,000 cells). If the ratio is 10:1, then there will be approximately 90909 effector cells and 9091 target cells (for a total of 100,000 cells). The total number of cells per well is not intended to limit the invention. The total number of cells per well can range from 10,000 to 200,000 but it is not so limited. In specific embodiments, the total number of cells per cell is at least 10,000, at least 20,000, at least 25,000, at least 50,000, at least 75,000, at lest 100,000, at least 125,000, at least 150,000, at least 175,000, and at least 200,000 cells per well. In other embodiments, the total number of cells per well may be as low as 10 cells, 100 cells, 500 cells, 1000 cells, or 5,000 cells. The total number of cells will depend upon the nature and activity of the effector cell, the target cell, or both. Those of ordinary skill in the art will be familiar with determining a suitable number of cells per well based on the characteristics of the cells involved and the volume and/or area of the chamber.

The assay is performed in a flat bottom chamber. Usually this will be a flat bottom well of a 96 well plate, with which the art is familiar. The volume in such a chamber is approximately 50 microliters. The flat bottom chamber however can also be an individual Petri dish, or a multiwell plate with fewer than 96 wells (e.g., a 6 well, 12 well, 24 well, or 48 well plate). The choice of chamber will depend upon the particular assay system and the ease of use. 96 well plates are generally preferred due to the ease of manipulation, and the ability to perform parallel assays simultaneously. The terms “chamber” and “well” are used interchangeably herein and refer to the container in which the effector and target cells are combined in order to perform the cytotoxicity assay of the invention. Usually, these chambers may include a removable lid in order to prevent evaporation of medium during incubation. The chambers may be of any shape provided they include a flat bottom. The sides of the chamber rising from the flat bottom may be made circular or arranged in a square or in a triangular configuration, but are not so limited. The volume of medium required will depend upon the configuration, and one of ordinary skill in the art will be able to routinely determine the necessary volume.

In one embodiment, the assay can include an (a) image processor and signal (e.g., image) capturing device, (b) computing device, which is coupled to the image processor, and (c) a database. The image processor receives information from the signal capturing device, which in turn acquires signals produced by cell lysis. For example, a signal capturing device according to the invention could be a digital camera which contains an automatic shutter for exposure control and is adapted to receive fluorescent light from a microspcope assembly set for visualizing cell lysis. Here, the digital camera could be in communication with a computing device, such as a desktop personal computer, via an image processor. The computing device facilitates the user to visualize, manipulate, analyze, render, and process, etc., the data generated by the methods of the present invention. The data can be stored and retrieved in a suitable database, which can be located on a local computing device, such as a computer hard drive, or over a network system on a remotely-located computer.

One of ordinary skill in the art will also appreciate that the data can also be transmitted to another another person, computing device, or destination via any known method of data transfer, including, for example portable storage media, network transfers, or by providing printed copies of data. Thus, oweing to the transferability of the data generated from the methods of the instant invention, especially the high throughput screening assays taught herein, those skilled in the art will appreciate that there can be a cooperation between a plurality of persons or research groups that are distally located from one another. For example, a first research group in a first global location could carry out a first segment of the high throughput methods of the instant invention whereas a second research group in a second global location in coordination therebetween could carry out a second segment of the high throughput methods of the invention. For example, the first segment carried out by a first research group might relate to generating the data from a cell-based screen of the present invention to identify test agents having desired activity and providing said data to an accessible database. The second segment carried out by the second research group might relate to the acquirement of the data from the database and analyzing said data to identify and further study test agents.

In one embodiment, the invention can be carried out using a microwell format in a microplate, provided that the well bottoms are flat. The microplate, such as one having 96-, 384-, or 1536-wells, could be placed in an “XY” microplate reader and the signal contained in each of the wells of the microwell plate could be detected by, for example, a digital camera or scintillation counter, and the data sent to a database. A computing device, such as a laptop computer, could retrieve the information from the assay and display the results thereon. Any known software and/or image processing technology is contemplated by the present invention for obtaining the results of the cell-based assays of the present invention, especially the fluorescence-based assays. Acquiring, processing, and storing of data and other assay-relevant data from high throughput cell-based screens is known in the art and can be found in U.S. Pat. Nos. 5,989,835, 6,631,331, 6,620,591, 6,633,818, and 6,416,959, wherein each of said patents is incorporated herein by reference in their entirety.

The present invention further contemplates any suitable future-developed instrumentation for measuring, acquiring, detecting, analyzing, processing, and storing the data generated from the screening methods of the instant invention. One of ordinary skill in the art will appreciate that instrumentation and technology to facilitate high throughput assays are continually being developed, such as improved fluorescence readers, robotics, bioinformatics, software, and assay reaction vessels. The present invention contemplates any such method suitable for carrying the instant invention.

It has been further been discovered, by carrying out methods of the invention, that the HIV gp120 protein causes fugetaxis of immune cells having cytotoxic activity, such as CTLs In particular, it has been shown that gp120 induces bidirectional movement of immune cells such as T cells. The migratory role of gp120 in this regard was heretofore unknown. gp120 was previously reported to have chemoattractant activity for both CD4+ and CD8+ cells. (Iyengar et al. 1999 J. Immunol 162:6263; Misse et al. 1999 Blood 93:2454.)

Depending upon its concentration, gp120 can stimulate chemotaxis of immune cells (i.e., movement of the immune cells towards gp120), or fugetaxis of immune cells (i.e., movement of the immune cells away from gp120), or chemokinesis of immune cells (i.e., random movement in response to gp120). Accordingly, gp120 stimulates chemotaxis of immune cells and at high concentrations, it stimulates fugetaxis of immune cells.

Thus, in one embodiment of the invention, gp120 and inhibitors of gp120 can be used to modulate immune responses, particularly with respect to stimulating or inhibiting movement of immune cells such as cytotoxic T lymphocytes.

The invention is therefore useful in inhibiting abnormal immune responses such as inappropriate or excessive immune responses. Accordingly, a method is provided for inhibiting an abnormal immune response comprising administering to a subject in need thereof a gp120 molecule or functional equivalent thereof in an amount effective to inhibit an abnormal immune response.

As used herein, “inhibit”, “inhibited” or “inhibiting” refers to a decrease of a property or activity of molecules or cells or response either to complete elimination or to a lower level.

As used herein, “modulate,” “modulated” or “modulating” refers to regulation of a property or activity of molecules or cells in a negative or positive manner.

As used herein, a “subject” includes a human, non-human primate, cow, horse, pig, sheep, goat, dog, cat or rodent. In all embodiments human subjects are preferred.

A “gp120 molecule” is a gp120 nucleic acid or a gp120 polypeptide or fragment thereof that retains the ability to stimulate chemotaxis, fugetaxis, or both. gp120 molecules include molecules encoding, or encoded by, both degenerate and non-degenerate variants of gp120 DNA sequences. Preferably, the gp120 molecule, or the functional equivalent thereof, is not antigenic. A functional equivalent of gp120 includes molecules sharing sequence similarity (e.g., homology or identity) with gp120 and that stimulate chemotaxis, fugetaxis, or both. Preferably, the sequence similarity comprises at least 75% amino acid sequence homology, and even more preferably comprises 80%, 85%, 90% or 95% amino acid sequence homology.

The abnormal immune response may be selected from the group consisting of autoimmune disease, inflammation, immune hypersensitivity, allergy, asthma, and graft-versus-host disease (GVHD). In important embodiments, the abnormal immune response is reduced to a normal level or eliminated completely. gp120 molecules can be used to tolerize the immune system to an antigen that it would otherwise mount an immune response against.

The abnormal immune response may involvement chemotaxis or fugetaxis of various immune cells including but not limited to CD4+ T cells, CD8+ T cells, neutrophils, macrophages, natural killer (NK) cells, dendritic cells, and the like. In one example, the abnormal immune response involves infiltration of T lymphocytes. In this latter example, the gp120 molecule inhibits the infiltration of T lymphocytes.

An example of an abnormal immune response is an autoimmune disease. “Autoimmune disease” as used herein, results when a subject's immune system attacks its own organs or tissues, producing a clinical condition associated with the destruction of that tissue, as exemplified by diseases such as uveitis, insulin-dependent diabetes mellitus, autoimmune hemolytic anemias, rheumatic fever, Crohn's disease, Guillain-Barre syndrome, psoriasis, thyroiditis, Graves' disease, myasthenia gravis, autoimmune hepatitis, multiple sclerosis, systemic lupus erythematosus, rheumatoid arthritis, autoimmune encephalomyelitis, Hashimoto's thyroiditis, Goodpasture's syndrome, pemphigus (e.g., pemphigus vulgaris), autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus, but it is not so limited.

Autoimmune disease may be caused by a genetic predisposition alone, by certain exogenous agents (e.g., viruses, bacteria, chemical agents, etc.), or both. Some forms of autoimmunity arise as the result of trauma to an area usually not exposed to lymphocytes, such as neural tissue or the lens of the eye. When the tissues in these areas become exposed to lymphocytes, their surface proteins can act as antigens and trigger the production of antibodies and cellular immune responses which then begin to destroy those tissues. Other autoimmune diseases develop after exposure of a subject to antigens which are antigenically similar to, that is cross-reactive with, the subject's own tissue. In rheumatic fever, for example, an antigen of the streptococcal bacterium, which causes rheumatic fever, is cross-reactive with parts of the human heart. The antibodies cannot differentiate between the bacterial antigens and the heart muscle antigens, consequently cells with either of those antigens can be destroyed.

Other autoimmune diseases, for example, insulin-dependent diabetes mellitus (involving the destruction of the insulin producing beta-cells of the islets of Langerhans), multiple sclerosis (involving the destruction of the conducting fibers of the nervous system) and rheumatoid arthritis (involving the destruction of the joint-lining tissue), are characterized as being the result of a mostly cell-mediated autoimmune response and appear to be due primarily to the action of T cells (See, Sinha et al., Science, 1990, 248:1380). Yet others, such as myesthenia gravis and systemic lupus erythematosus, are characterized as being the result of primarily a humoral autoimmune response. In some embodiments, the subject has rheumatoid arthritis, multiple sclerosis, or uveitis.

Another example of an abnormal immune response is graft versus host disease.

The invention provides a method of inhibiting migration of immune cells to a site of inflammation in the subject. “Inflammation” as used herein, is a localized protective response elicited by a foreign (non-self) antigen, and/or by an injury or destruction of tissue(s), which serves to destroy, dilute or sequester the foreign antigen, the injurious agent, and/or the injured tissue. Inflammation occurs when tissues are injured by viruses, bacteria, trauma, chemicals, heat, cold, or any other harmful stimuli. In such instances, the classic weapons of the immune system (T cells, B cells, macrophages) interface with cells and soluble products that are mediators of inflammatory responses (neutrophils, eosinophils, basophils, kinin and coagulation systems, and complement cascade).

Inflammation is typically characterized by (i) migration of leukocytes at the site of antigen (injury) localization; (ii) specific and nonspecific recognition of “foreign” and other (necrotic/injured tissue) antigens mediated by B and T lymphocytes, macrophages and the alternative complement pathway; (iii) amplification of the inflammatory response with the recruitment of specific and nonspecific effector cells by complement components, lymphokines and monokines, kinins, arachidonic acid metabolites, and mast cell/basophil products; and (iv) macrophage, neutrophil and lymphocyte participation in antigen destruction with ultimate removal of antigen particles (injured tissue) by phagocytosis.

In further embodiments, the inflammation is caused by an immune response against “non-self-antigens” (including antigens of necrotic self-material), and the subject in need of treatment according to the invention is a transplant recipient, has atherosclerosis, has suffered a myocardial infarction and/or an ischemic stroke, has an abscess, and/or has myocarditis. This is because after cell (or organ) transplantation, or after myocardial infarction or ischemic stroke, certain antigens from the transplanted cells (organs), or necrotic cells from the heart or the brain, can stimulate the production of immune lymphocytes and/or autoantibodies, which later participate in inflammation/rejection (in the case of a transplant), or attack cardiac or brain target cells causing inflammation and aggravating the condition (Johnson et al., Sem. Nuc. Med. 1989, 19:238; Leinonen et al., Microbiol. Path., 1990, 9:67; Montalban et al., Stroke, 1991, 22:750).

Inflammatory conditions include, but are not limited to, autoimmune disorders, psoriasis, rheumatoid arthritis, experimental autoimmune encephalomyelitis (EAE), Crohn's disease, ulcerative colitis, allergic inflammatory diseases, such as asthma, excema, contact dermatitis, latex dermatitis, inflammatory bowel disease, anathylaxis, allergic rhinitis (hayfever), atopic dermatitis, graft versus host disease, and multiple sclerosis.

An “allergy” refers to acquired hypersensitivity to a substance (allergen). A “subject having an allergy” is a subject that has an allergic reaction in response to an allergen.

The allergic reaction has been extensively studied and the basic immune mechanisms involved are well known. Allergic conditions or diseases in humans include but are not limited to eczema, allergic rhinitis or coryza, hay fever, conjunctivitis, bronchial or allergic asthma, urticaria (hives) and food allergies; atopic dermatitis; anaphylaxis; drug allergy; angioedema; and allergic conjunctivitis. Allergic diseases in dogs include but are not limited to seasonal dermatitis; perennial dermatitis; rhinitis: conjunctivitis; allergic asthma; and drug reactions. Allergic diseases in cats include but are not limited to dermatitis and respiratory disorders; and food allergens. Allergic diseases in horses include but are not limited to respiratory disorders such as “heaves” and dermatitis. Allergic diseases in non-human primates include but are not limited to allergic asthma and allergic dermatitis.

The generic name for molecules that cause an allergic reaction is allergen. There are numerous species of allergens. The allergic reaction occurs when tissue-sensitizing immunoglobulin of the IgE type reacts with foreign allergen. The IgE antibody is bound to mast cells and/or basophils, and these specialized cells release chemical mediators (vasoactive amines) of the allergic reaction when stimulated to do so by allergens bridging the ends of the antibody molecule. Histamine, platelet activating factor, arachidonic acid metabolites, and serotonin are among the best known mediators of allergic reactions in man. Histamine and the other vasoactive amines are normally stored in mast cells and basophil leukocytes. The mast cells are dispersed throughout animal tissue and the basophils circulate within the vascular system. These cells manufacture and store histamine within the cell unless the specialized sequence of events involving IgE binding occurs to trigger its release.

The symptoms of the allergic reaction vary, depending on the location within the body where the IgE reacts with the antigen. If the reaction occurs along the respiratory epithelium the symptoms are sneezing, coughing and asthmatic reactions. If the interaction occurs in the digestive tract, as in the case of food allergies, abdominal pain and diarrhea are common. Systematic reactions, for example following a bee sting, can be severe and often life threatening.

Delayed type hypersensitivity, also known as type IV allergy reaction is an allergic reaction characterized by a delay period of at least 12 hours from invasion of the antigen into the allergic subject until appearance of the inflammatory or immune reaction. The T lymphocytes (sensitized T lymphocytes) of individuals in an allergic condition react with the antigen, triggering the T lymphocytes to release lymphokines (macrophage migration inhibitory factor (MIF), macrophage activating factor (MAF), mitogenic factor (MF), skin-reactive factor (SRF), chemotactic factor, neovascularization-accelerating factor, etc.), which function as inflammation mediators, and the biological activity of these lymphokines, together with the direct and indirect effects of locally appearing lymphocytes and other inflammatory immune cells, give rise to the type IV allergy reaction. Delayed allergy reactions include tuberculin type reaction, homograft rejection reaction, cell-dependent type protective reaction, contact dermatitis hypersensitivity reaction, and the like, which are known to be most strongly suppressed by steroidal agents. Consequently, steroidal agents are effective against diseases which are caused by delayed allergy reactions. Long-term use of steroidal agents at concentrations currently being used can, however, lead to the serious side-effect known as steroid dependence. The methods of the invention solve some of these problems, by providing for lower and fewer doses to be administered.

Immediate immune hypersensitivity (or anaphylactic response) is a form of allergic reaction which develops very quickly, i.e. within seconds or minutes of exposure of the patient to the causative allergen, and it is mediated by IgE antibodies made by B lymphocytes. In nonallergic patients, there is no IgE antibody of clinical relevance; but, in a person suffering with allergic diseases, IgE antibody mediates immediate hypersensitivity by sensitizing mast cells which are abundant in the skin, lymphoid organs, in the membranes of the eye, nose and mouth, and in the respiratory tract and intestines.

Mast cells have surface receptors for IgE, and the IgE antibodies in allergy-suffering patients become bound to them. As discussed briefly above, when the bound IgE is subsequently contacted by the appropriate allergen, the mast cell is caused to degranulate and to release various substances called bioactive mediators, such as histamine, into the surrounding tissue. It is the biologic activity of these substances which is responsible for the clinical symptoms typical of immediate hypersensitivity; namely, contraction of smooth muscle in the airways or the intestine, the dilation of small blood vessels and the increase in their permeability to water and plasma proteins, the secretion of thick sticky mucus, and in the skin, redness, swelling and the stimulation of nerve endings that results in itching or pain.

Many allergies are caused by IgE antibody generation against harmless allergens. The types of antibodies associated with a Th1 response are generally more protective because they have high neutralization and opsonization capabilities. Th2 responses involve predominately antibodies and these have less protective effect against infection and some Th2 isotypes (e.g., IgE) are associated with allergy. Strongly polarized Th1 and Th2 responses not only play different roles in protection, they can promote different immunopathological reactions. Th1-type responses are involved organ specific autoimmunity such as experimental autoimmune uveoretinitis (Dubey et al, 1991, Eur Cytokine Network 2: 147-152), experimental autoimmune encephalitis (EAE) (Beraud et al, 1991, Cell Immunol 133: 379-389) and insulin dependent diabetes mellitus (Hahn et al, 1987, Eur. J. Immunol. 18: 2037-2042), in contact dermatitis (Kapsenberg et al, Immunol Today 12: 392-395), and in some chronic inflammatory disorders. In contrast Th2-type responses are responsible for triggering allergic atopic disorders (against common environmental allergens) such as allergic asthma (Walker et al, 1992, Am Rev Resp Dis 148: 109-115) and atopic dermatitis (van der Heijden et al, 1991, J Invest Derm 97: 389-394), are thought to exacerbate infection with tissue-dwelling protozoa such as helminths (Finkelman et al, 1991, Immunoparasitol Today 12: A62-66) and Leishmania major (Caceres-Dittmar et al, 1993, Clin Exp Immunol 91: 500-505), are preferentially induced in certain primary immunodeficiencies such as hyper-IgE syndrome (Del Prete et al, 1989, J Clin Invest 84: 1830-1835) and Omenn's syndrome (Schandene et al, 1993, Eur J Immunol 23: 56-60), and are associated with reduced ability to suppress HIV replication (Barker et al, 1995, Proc Soc Nat Acad Sci USA 92: 11135-11139).

Thus, in general, it appears that allergic diseases are mediated by Th2 type immune responses. Th2 cytokines, especially IL-4 and IL-5 are elevated in the airways of asthmatic subjects. These cytokines promote important aspects of the asthmatic inflammatory response, including IgE isotype switching, eosinophil chemotaxis and activation, and mast cell growth. Th1 cytokines, especially IFN-g and IL-12, can suppress the formation of Th2 clones and production of Th2 cytokines.

An “allergen” as used herein is a molecule capable of provoking an immune response characterized by production of IgE. Thus, in the context of this invention, the term allergen means a specific type of antigen which can trigger an allergic response which is mediated by IgE antibody. The method and preparations of this invention extend to a broad class of such allergens and fragments of allergens or haptens acting as allergens. Allergens include but are not limited to Environmental Aeroallergens; plant pollens such as Ragweed/hayfever; Weed pollen allergens; Grass pollen allergens; Johnson grass; Tree pollen allergens; Ryegrass; House dust mite allergens; Storage mite allergens; Japanese cedar pollen/hay fever Mold spore allergens; Animal allergens (cat, dog, guinea pig, hamster, gerbil, rat, mouse); Food Allergens (e.g., Crustaceans; nuts, such as peanuts; citrus fruits); Insect Allergens (Other than mites listed above); Venoms: (Hymenoptera, yellow jacket, honey bee, wasp, hornet, fire ant); Other environmental insect allergens from cockroaches, fleas, mosquitoes, etc.; Bacteria such as streptococcal antigens; Parasites such as Ascaris antigen; Viral Antigens; Fungal spores; Drug Allergens; Antibiotics; penicillins and related compounds; other antibiotics; Whole Proteins such as hormones (insulin), enzymes (Streptokinase); all drugs and their metabolites capable of acting as incomplete antigens or haptens; Industrial Chemicals and metabolites capable of acting as haptens and stimulating the immune system (Examples are the acid anhydrides (such as trimellitic anhydride) and the isocyanates (such as toluene diisocyanate)); Occupational Allergens such as flour (i.e. Baker's asthina), castor bean, coffee bean, and industrial chemicals described above; flea allergens; and human proteins in non-human animals.

Allergens include but are not limited to cells, cell extracts, proteins, polypeptides, peptides, polysaccharides, polysaccharide conjugates, peptide and non-peptide mimics of polysaccharides and other molecules, small molecules, lipids, glycolipids, and carbohydrates. Many allergens, however, are protein or polypeptide in nature, as proteins and polypeptides are generally more antigenic than carbohydrates or fats.

Examples of specific natural, animal and plant allergens include but are not limited to proteins specific to the following genuses: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

Asthma is a chronic inflammatory disease which manifests symptoms of recurrent episodes of wheezing, breathlessness, and chest tightness, and coughing, resulting from airflow obstruction. Airway inflammation associated with asthma can be detected through observation of a number of physiological changes, such as, denudation of airway epithelium, collagen deposition beneath basement membrane, edema, mast cell activation, inflammatory cell infiltration, including neutrophils, eosinophils, and lymphocytes. As a result of the airway inflammation, asthma patients often experience airway hyper-responsiveness, airflow limitation, respiratory symptoms, and disease chronicity. Airflow limitations include acute bronchoconstriction, airway edema, mucous plug formation, and airway remodeling, features which often lead to bronchial obstruction. In some cases of asthma, subbasement membrane fibrosis may occur, leading to persistent abnormalities in lung function.

A “subject having asthma” is a subject that has a disorder of the respiratory system characterized by inflammation, narrowing of the airways and increased reactivity of the airways to inhaled agents. Asthma is frequently, although not exclusively associated with atopic or allergic symptoms. An “initiator” as used herein refers to a composition or environmental condition which triggers asthma. Initiators include, but are not limited to, allergens, cold temperatures, exercise, viral infections, SO₂.

In another aspect the invention provides methods for treating or preventing asthma or allergy in a hypo-responsive subject. As used herein, a hypo-responsive subject is one who has previously failed to respond to a treatment directed at treating or preventing asthma or allergy or one who is at risk of not responding to such a treatment. The treatment directed at treating or preventing asthma or allergy may be an asthma/allergy medicament, in which case the hypo-responsive subject is one who is hypo-responsive to an asthma/allergy medicament. Other subjects who are hypo-responsive include those who are refractory to an asthma/allergy medicament. As used herein, the term “refractory” means resistant or failure to yield to treatment. Such subjects may be those who never responded to an asthma/allergy medicament (i.e., subjects who are non-responders), or alternatively, they may be those who at one time responded to an asthma/allergy medicament, but have since that time have become refractory to the medicament. In some embodiments, the subject is one who is refractory to a subset of medicaments. A subset of medicaments is at least one medicament. In some embodiments, a subset refers to 2, 3, 4, 5, 6, 7, 8, 9, or 10 medicaments.

In other embodiments, hypo-responsive subjects are elderly subjects, regardless of whether they have or have not previously responded to a treatment directed at treating or preventing asthma or allergy. Elderly subjects, even those who have previously responded to such treatment, are considered to be at risk of not responding to a future administration of this treatment. Similarly, neonatal subjects are also considered to be at risk of not responding to treatment directed at treating or preventing asthma or allergy.

An “asthma/allergy medicament” as used herein is a composition of matter which reduces the symptoms, inhibits the asthmatic or allergic reaction, or prevents the development of an allergic or asthmatic reaction. Various types of medicaments for the treatment of asthma and allergy are described in the Guidelines For The Diagnosis and Management of Asthma, Expert Panel Report 2, NIH Publication No. 97/4051, Jul. 19, 1997, the entire contents of which are incorporated herein by reference. The summary of the medicaments as described in the NIH publication is presented below.

In most embodiments the asthma/allergy medicament is useful to some degree for treating both asthma and allergy. Some asthma/allergy medicaments are preferably used in combination with the gp120 molecules to treat asthma These are referred to as asthma medicaments. Asthma medicaments include, but are not limited, PDE-4 inhibitors, bronchodilator/beta-2 agonists, K+ channel openers, VLA-4 antagonists, neurokin antagonists, TXA2 synthesis inhibitors, xanthanines, arachidonic acid antagonists, 5 lipoxygenase inhibitors, thromboxin A2 receptor antagonists, thromboxane A2 antagonists, inhibitor of 5-lipox activation proteins, and protease inhibitors.

Bronchodilator/beta-2 agonists are a class of compounds which cause bronchodilation or smooth muscle relaxation. Bronchodilator/beta-2 agonists include, but are not limited to, salmeterol, salbutamol, albuterol, terbutaline, D2522/formoterol, fenoterol, bitolterol, pirbuerol methylxanthines and orciprenaline. Long-acting β₂ agonists and bronchodilators are compounds which are used for long-term prevention of symptoms in addition to the anti-inflammatory therapies. They function by causing bronchodilation, or smooth muscle relaxation, following adenylate cyclase activation and increase in cyclic AMP producing functional antagonism of bronchoconstriction. These compounds also inhibit mast cell mediator release, decrease vascular permeability and increase mucociliary clearance. Long-acting β₂ agonists include, but are not limited to, salmeterol and albuterol. These compounds are usually used in combination with corticosteroids and generally are not used without any inflammatory therapy. They have been associated with side effects such as tachycardia, skeletal muscle tremor, hypokalemia, and prolongation of QTc interval in overdose.

Methylxanthines, including for instance theophylline, have been used for long-term control and prevention of symptoms. These compounds cause bronchodilation resulting from phosphodiesterase inhibition and likely adenosine antagonism. It is also believed that these compounds may effect eosinophilic infiltration into bronchial mucosa and decrease T-lymphocyte numbers in the epithelium. Dose-related acute toxicities are a particular problem with these types of compounds. As a result, routine serum concentration must be monitored in order to account for the toxicity and narrow therapeutic range arising from individual differences in metabolic clearance. Side effects include tachycardia, nausea and vomiting, tachyarrhythmias, central nervous system stimulation, headache, seizures, hematemesis, hyperglycemia and hypokalemia. Short-acting β₂ agonists/bronchodilators relax airway smooth muscle, causing the increase in air flow. These types of compounds are a preferred drug for the treatment of acute asthmatic systems. Previously, short-acting β₂ agonists had been prescribed on a regularly-scheduled basis in order to improve overall asthma symptoms. Later reports, however, suggested that regular use of this class of drugs produced significant diminution in asthma control and pulmonary function (Sears, et al. Lancet; 336:1391-6, 1990). Other studies showed that regular use of some types of β₂ agonists produced no harmful effects over a four-month period but also produced no demonstrable effects (Drazen, et al., N. Eng. J. Med.; 335:841-7, 1996). As a result of these studies, the daily use of short-acting β₂ agonists is not generally recommended. Short-acting β₂ agonists include, but are not limited to, albuterol, bitolterol, pirbuterol, and terbutaline. Some of the adverse effects associated with the mastration of short-acting β₂ agonists include tachycardia, skeletal muscle tremor, hypokalemia, increased lactic acid, headache, and hyperglycemia.

Other asthma/allergy medicaments are preferably used in combination with the gp120 molecules to treat allergy. These are referred to as allergy medicaments. Allergy medicaments include, but are not limited to, anti-histamines, steroids, and prostaglandin inducers. Anti-histamines are compounds which counteract histamine released by mast cells or basophils. These compounds are well known in the art and commonly used for the treatment of allergy. Anti-histamines include, but are not limited to, loratidine, cetirizine, buclizine, ceterizine analogues, fexofenadine, terfenadine, desloratadine, norastemizole, epinastine, ebastine, ebastine, astemizole, levocabastine, azelastine, tranilast, terfenadine, mizolastine, betatastine, CS 560, and HSR 609. Prostaglandin inducers are compounds which induce prostaglandin activity. Prostaglandins function by regulating smooth muscle relaxation. Prostaglandin inducers include, but are not limited to, S-5751.

The asthma/allergy medicaments useful also include steroids and immunomodulators.

The steroids include, but are not limited to, beclomethasone, fluticasone, tramcinolone, budesonide, corticosteroids and budesonide.

Corticosteroids are used long-term to prevent development of the symptoms, and suppress, control, and reverse inflammation arising from an initiator. Some corticosteroids can be administered by inhalation and others are administered systemically. The corticosteroids that are inhaled have an anti-inflammatory function by blocking late-reaction allergen and reducing airway hyper-responsiveness. These drugs also inhibit cytokine production, adhesion protein activation, and inflammatory cell migration and activation.

Corticosteroids include, but are not limited to, beclomethasome dipropionate, budesonide, flunisolide, fluticaosone, propionate, and triamcinoone acetonide. Although dexamethasone is a corticosteroid having anti-inflammatory action, it is not regularly used for the treatment of asthma/allergy in an inhaled form because it is highly absorbed, it has long-term suppressive side effects at an effective dose. Dexamethasone, however, can be used according to the invention for the treating of asthma/allergy because when administered in combination with gp120 molecules it can be administered at a low dose to reduce the side effects. Some of the side effects associated with corticosteroid include cough, dysphonia, oral thrush (candidiasis), and in higher doses, systemic effects, such as adrenal suppression, osteoporosis, growth suppression, skin thinning and easy bruising. (Barnes & Peterson, Am. Rev. Respir. Dis.; 148:S1-S26, 1993; and Kamada et al., Am. J Respir. Crit. Care Med.; 153:1739-48, 1996)

Systemic corticosteroids include, but are not limited to, methylprednisolone, prednisolone and prednisone. Cortosteroids are used generally for moderate to severe exacerbations to prevent the progression, reverse inflammation and speed recovery. These anti-inflammatory compounds include, but are not limited to, methylprednisolone, prednisolone, and prednisone. Cortosteroids are associated with reversible abnormalities in glucose metabolism, increased appetite, fluid retention, weight gain, mood alteration, hypertension, peptic ulcer, and rarely asceptic necrosis of femur. These compounds are useful for short-term (3-10 days) prevention of the inflammatory reaction in inadequately controlled persistent asthma. They also function in a long-term prevention of symptoms in severe persistent asthma to suppress and control and actually reverse inflammation. The side effects associated with systemic corticosteroids are even greater than those associated with inhaled corticosteroids. Side effects include, for instance, reversible abnormalities in glucose metabolism, increased appetite, fluid retention, weight gain, mood alteration, hypertension, peptic ulcer and asceptic necrosis of femur, which are associated with short-term use. Some side effects associated with longer term use include adrenal axis suppression, growth suppression, dermal thinning, hypertension, diabetes, Cushing's syndrome, cataracts, muscle weakness, and in rare instances, impaired immune function. It is recommended that these types of compounds be used at their lowest effective dose (guidelines for the diagnosis and management of asthma; expert panel report to; NIH Publication No. 97-4051; July 1997). The inhaled corticosteroids are believed to function by blocking late reaction to allergen and reducing airway hyper-responsiveness. Their also believed to reverse β₂-receptor downregulation and to inhibit microvascular leakage.

The immunomodulators include, but are not limited to, the group consisting of anti-inflammatory agents, leukotriene antagonists, IL-4 muteins, soluble IL-4 receptors, immunosuppressants (such as tolerizing peptide vaccine), anti-IL-4 antibodies, IL-4 antagonists, anti-IL-5 antibodies, soluble IL-13 receptor-Fc fusion proteins, anti-IL-9 antibodies, CCR3 antagonists, CCR5 antagonists, VLA-4 inhibitors, and, and downregulators of IgE.

Leukotriene modifiers are often used for long-term control and prevention of symptoms in mild persistent asthma. Leukotriene modifiers function as leukotriene receptor antagonists by selectively competing for LTD-4 and LTE-4 receptors. These compounds include, but are not limited to, zafirlukast tablets and zileuton tablets. Zileuton tablets function as 5-lipoxygenase inhibitors. These drugs have been associated with the elevation of liver enzymes and some cases of reversible hepatitis and hyperbilirubinemia. Leukotrienes are biochemical mediators that are released from mast cells, eosinophils, and basophils that cause contraction of airway smooth muscle and increase vascular permeability, mucous secretions and activate inflammatory cells in the airways of patients with asthma.

Other immunomodulators include neuropeptides that have been shown to have immunomodulating properties. Functional studies have shown that substance P, for instance, can influence lymphocyte function by specific receptor mediated mechanisms. Substance P also has been shown to modulate distinct immediate hypersensitivity responses by stimulating the generation of arachidonic acid-derived mediators from mucosal mast cells. J. McGillies, et al., Substance P and Immunoregulation, Fed. Proc. 46:196-9 (1987). Substance P is a neuropeptide first identified in 1931 by Von Euler and Gaddum. An unidentified depressor substance in certain tissue extracts, J. Physiol. (London) 72:74-87 (1931). Its amino acid sequence, Arg-Pro-Lys-Pro-Gln-Gln-Phe-Phe-Gly-Leu-Met-NH.sub.2 (Sequence Id. No. 1) was reported by Chang et al. in 1971. Amino acid sequence of substance P, Nature (London) New Biol. 232:86-87 (1971). The immunoregulatory activity of fragments of substance P has been studied by Siemion, et al. Immunoregulatory Activity of Substance P Fragments, Molec. Immunol. 27:887-890 (1990).

Another class of compounds is the down-regulators of IgE. These compounds include peptides or other molecules with the ability to bind to the IgE receptor and thereby prevent binding of antigen-specific IgE. Another type of downregulator of IgE is a monoclonal antibody directed against the IgE receptor-binding region of the human IgE molecule. Thus, one type of downregulator of IgE is an anti-IgE antibody or antibody fragment. Anti-IgE is being developed by Genentech. One of skill in the art could prepare functionally active antibody fragments of binding peptides which have the same function. Other types of IgE downregulators are polypeptides capable of blocking the binding of the IgE antibody to the Fc receptors on the cell surfaces and displacing IgE from binding sites upon which IgE is already bound.

One problem associated with downregulators of IgE is that many molecules don't have a binding strength to the receptor corresponding to the very strong interaction between the native IgE molecule and its receptor. The molecules having this strength tend to bind irreversibly to the receptor. However, such substances are relatively toxic since they can bind covalently and block other structurally similar molecules in the body. Of interest in this context is that the alpha chain of the IgE receptor belongs to a larger gene family where i.e. several of the different IgG Fc receptors are contained. These receptors are absolutely essential for the defense of the body against i.e. bacterial infections. Molecules activated for covalent binding are, furthermore, often relatively unstable and therefore they probably have to be administered several times a day and then in relatively high concentrations in order to make it possible to block completely the continuously renewing pool of IgE receptors on mast cells and basophilic leukocytes.

These types of asthma/allergy medicaments are sometimes classified as long-term control medications or quick-relief medications. Long-term control medications include compounds such as corticosteroids (also referred to as glucocorticoids), methylprednisolone, prednisolone, prednisone, cromolyn sodium, nedocromil, long-acting β₂-agonists, methylxanthines, and leukotriene modifiers. Quick relief medications are useful for providing quick relief of symptoms arising from allergic or asthmatic responses. Quick relief medications include short-acting β₂ agonists, anticholinergics and systemic corticosteroids.

Chromolyn sodium and medocromil are used as long-term control medications for preventing primarily asthma symptoms arising from exercise or allergic symptoms arising from allergens. These compounds are believed to block early and late reactions to allergens by interfering with chloride channel function. They also stabilize mast cell membranes and inhibit activation and release of mediators from eosinophils and epithelial cells. A four to six week period of administration is generally required to achieve a maximum benefit.

Anticholinergics are generally used for the relief of acute bronchospasm. These compounds are believed to function by competitive inhibition of muscarinic cholinergic receptors. Anticholinergics include, but are not limited to, ipratrapoium bromide. These compounds reverse only cholinerigically-mediated bronchospasm and do not modify any reaction to antigen. Side effects include drying of the mouth and respiratory secretions, increased wheezing in some individuals, blurred vision if sprayed in the eyes.

In addition to standard asthma/allergy medicaments other methods for treating asthma/allergy have been used either alone or in combination with established medicaments. One preferred, but frequently impossible, method of relieving allergies is allergen or initiator avoidance. Another method currently used for treating allergic disease involves the injection of increasing doses of allergen to induce tolerance to the allergen and to prevent further allergic reactions.

Allergen injection therapy (allergen immunotherapy) is known to reduce the severity of allergic rhinitis. This treatment has been theorized to involve the production of a different form of antibody, a protective antibody which is termed a “blocking antibody”. Cooke, R A et al., Serologic Evidence of Immunity with Coexisting Sensitization in a Type of Human Allergy, Exp. Med. 62:733 (1935). Other attempts to treat allergy involve modifying the allergen chemically so that its ability to cause an immune response in the patient is unchanged, while its ability to cause an allergic reaction is substantially altered.

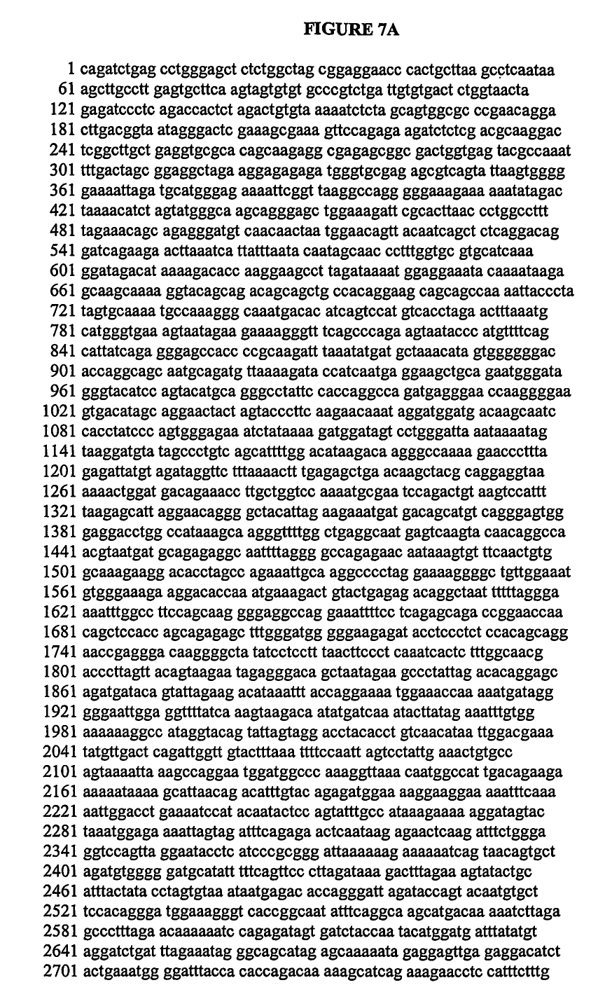

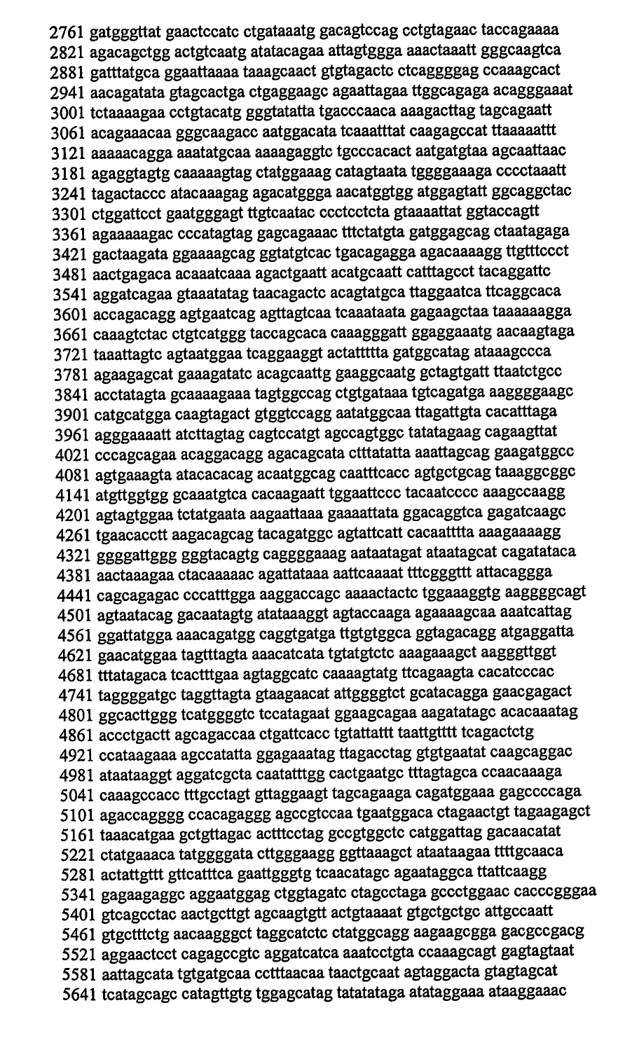

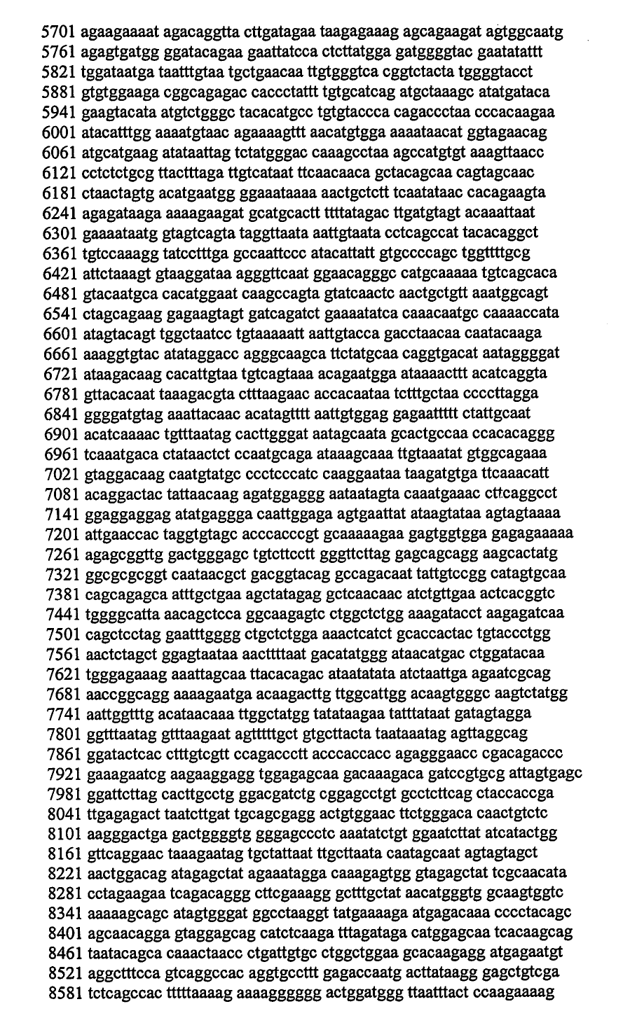

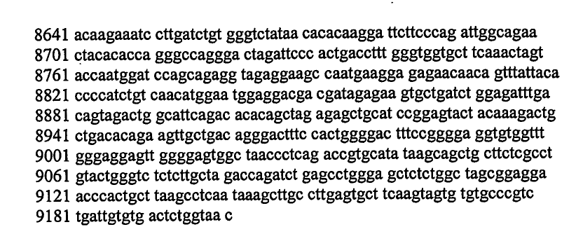

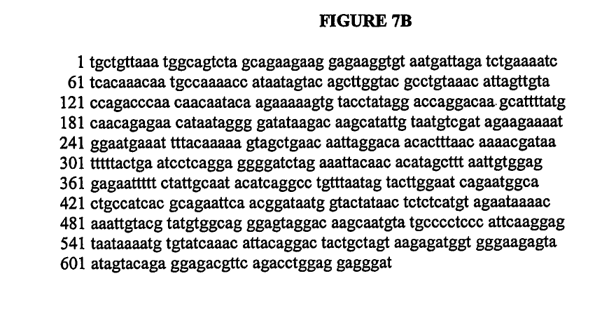

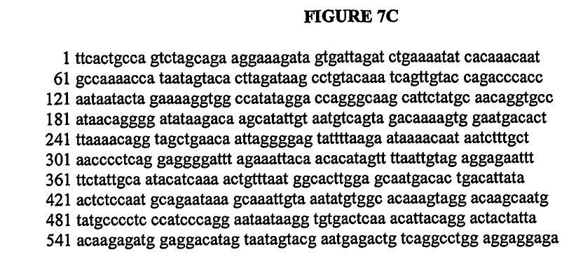

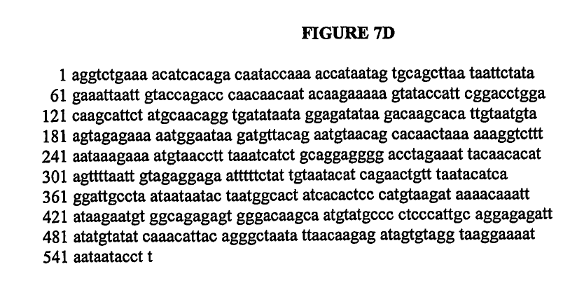

The abnormal response is inhibiting by administering a gp120 molecule. gp120, together with gp41, is encoded by the env gene of HIV. Exemplary nucleotide and amino acid sequences of gp120 are provided in FIG. 7. Others will be known to those of ordinary skill, and can be used interchangeably in the methods of the invention. gp120 is commercially available from a number of sources including Austral Biologicals. Recombinant gp120 can also be obtained from the AIDS Research and Reference Reagent Program.

The gp120 molecule can be used in soluble form, or it can be used in a cell bound form. A soluble gp120 is a gp120 polypeptide that is not cell associated. It may be conjugated to other agents such as therapeutic agents of imaging agents such as detectable labels. A cell bound form of gp120 is a gp120 polypeptide that is expressed by or attached to a cell. When used as a cell bound form, the gp120 nucleic acid may be transfected into a cell of interest under suitable transcriptional control elements, thereby allowing its expression on the surface of the cell. Those of ordinary skill in the art will be familiar with methods for transfection and expression of nucleic acids in cells. Cell bound forms of gp120 can be used in transplant settings, where cells or tissues to be transplanted can be transfected prior to transplant into a subject in order to reduce the likelihood of graft-versus-host disease (GVHD). This latter embodiment can also be used in stem cell transplants. Similarly, gp120 molecules can be used to tolerize the immune system to an antigen that it would otherwise mount an immune response against.

The gp120 molecule may be administered to cells via transfection or other nucleic acid delivery techniques known to those of ordinary skill (including electroporation and viral infection). The gp120 nucleic acid is generally provided in the context of a vector. As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Especially, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding PARG polypeptide or fragment or variant thereof. That heterologous DNA (RNA) is placed under operable control of transcriptional elements to permit the expression of the heterologous DNA in the host cell.

Preferred systems for mRNA expression in mammalian cells are those such as pRc/CMV (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen), which contains an Epstein Barr virus (EBV) origin of replication, facilitating the maintenance of plasmid as a multicopy extrachromosomal element. Another expression vector is the pEF-BOS plasmid containing the promoter of polypeptide Elongation Factor 1α, which stimulates efficiently transcription in vitro. The plasmid is described by Mishizuma and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno.P1A recombinant is disclosed by Warnier et al., in intradermal injection in mice for immunization against P1A (Int. J. Cancer, 67:303-310, 1996).

The invention in some aspects uses isolated gp120 protein. As used herein, with respect to polypeptides, “isolated” means separated from its native environment and present in sufficient quantity to permit its identification or use. Isolated, when referring to a protein or polypeptide, means, for example: (i) selectively produced by expression cloning or (ii) purified as by chromatography or electrophoresis. Isolated proteins or polypeptides may, but need not be, substantially pure.

A variety of methodologies well-known to the skilled practitioner can be utilized to obtain isolated gp120 protein. The polypeptide may be purified from cells or viruses which naturally produce the polypeptide by chromatographic means or immunological recognition. Alternatively, an expression vector may be introduced into cells to cause production of the polypeptide. In another method, mRNA transcripts may be microinjected or otherwise introduced into cells to cause production of the encoded polypeptide. Translation of mRNA in cell-free extracts such as the reticulocyte lysate system also may be used to produce polypeptide. Those skilled in the art also can readily follow known methods for isolating gp120 polypeptides. These include, but are not limited to, immunochromatography, HPLC, size-exclusion chromatography, ion-exchange chromatography and immune-affinity chromatography.

The term “substantially pure” means that the nucleic acid or protein/peptide is essentially free of other substances with which it may be found in nature or in vitro systems, to an extent practical and appropriate for their intended use. Substantially pure polypeptides may be produced by techniques well known in the art. As an example, because an isolated protein may be admixed with a pharmaceutically acceptable carrier in a pharmaceutical preparation, the protein may comprise only a small percentage by weight of the preparation. The protein is nonetheless isolated in that it has been separated from many of the substances with which it may be associated in living systems, i.e. isolated from certain other proteins.

The invention involves the use of isolated polypeptides, including whole proteins, partial proteins (e.g., domains) and peptide fragments (e.g., a fugetaxis inducing peptides or chemotactic inducing peptides of gp120). Fragments of a polypeptide preferably are those fragments that retain a distinct functional capability of the polypeptide, which in this case is the ability to stimulate chemotaxis, fugetaxis, or both. Such polypeptides are useful, for example, alone or as fusion proteins to generate antibodies, as targets for screening compounds for immunomodulatory reagents that bind gp120, as components of an immunoassay or diagnostic assay or as therapeutics. gp120 polypeptides can be isolated from biological samples including tissue, cell, or viral homogenates, and can also be expressed recombinantly in a variety of prokaryotic and eukaryotic expression systems by constructing an expression vector appropriate to the expression system, introducing the expression vector into the expression system, and isolating the recombinantly expressed protein. Short polypeptides, including peptides such as the aforementioned fugetaxis inducing or chemotaxis inducing peptides of gp120 can be synthesized chemically using well-established methods of peptide synthesis.

The invention also uses variants of the gp120 polypeptides described above. As used herein, a “variant” of a gp120 polypeptide is a polypeptide which contains one or more modifications to the primary amino acid sequence of a gp120 polypeptide. Modifications which create a gp120 polypeptide variant can be made to a gp120 polypeptide 1) to reduce or eliminate an activity of a gp120 polypeptide (i.e., its ability to be bound by HIV); 2) to enhance a property of a gp120 polypeptide, such as protein stability in an expression system or the stability of protein-protein binding; or 3) to provide a novel activity or property to a gp120 polypeptide, such as addition of a detectable moiety (such as the green fluorescent protein (GFP) fusions). Modifications to a gp120 polypeptide can be made to the nucleic acid which encodes the gp120 polypeptide, and can include deletions, point mutations, truncations, amino acid substitutions and additions of amino acids or non-amino acid moieties. Alternatively, modifications can be made directly to the polypeptide, such as by cleavage, addition of a linker molecule, addition of a detectable moiety, such as biotin or GFP, addition of a fatty acid, and the like. Modifications also embrace fusion proteins comprising all or part of the gp120 amino acid sequence.

The skilled artisan will also realize that conservative amino acid substitutions may be made in gp120 polypeptides to provide functionally equivalent variants of the foregoing polypeptides, i.e., the variants retain the functional capabilities of the gp120 polypeptides. As used herein, a “conservative amino acid substitution” refers to an amino acid substitution which does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants of the gp120 polypeptides include conservative amino acid substitutions of in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Conservative amino-acid substitutions in the amino acid sequence of gp120 polypeptides to produce functionally equivalent variants of gp120 polypeptides typically are made by alteration of a nucleic acid encoding a gp120 polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a gp120 polypeptide. Where amino acid substitutions are made to a small peptide fragment of gp120, the substitutions can be made by directly synthesizing the peptide. The activity of functionally equivalent fragments of gp120 polypeptides can be tested by cloning the gene encoding the altered gp120 polypeptide into a bacterial or mammalian expression vector, introducing the vector into an appropriate host cell, expressing the altered gp120 polypeptide, and testing for a functional capability of the gp120 polypeptides as disclosed herein. Peptides which are chemically synthesized can be tested directly for function, e.g., for binding to antisera recognizing associated antigens.

Preferably, the gp120 molecule, or the functional equivalent thereof, is not antigenic. Several references describe the generation of immunogenic fragments of gp120, and therefore, one skilled in the art would understand how to avoid these fragments, either by deletion, modification or otherwise, in one or embodiments of the invention. See, for example, Kim et al., 2003, Virology 305:124-137 and U.S. Pat. No. 6,585,979, the contents of which are incorporated herein by reference for their description of immunogenic gp120 fragments.

As used herein, the terms protein and polypeptide are used interchangeably.

The invention also provides methods for targeting gp120 with gp120 inhibitors. A gp120 inhibitor is an agent that inhibits the fugetactic or chemotactic activity of gp120, thereby modulating the movement of immune cells. A gp120 inhibitor may act directly upon gp120 by preventing its interaction with its ligands, or it may act upstream or downstream of gp120.

The chemotactic, fugetactic or chemokinetic response can be measured as described herein, or according to the transmigration assays described in greater detail in U.S. Pat. No. 6,448,054 B1, and in U.S. Pat. No. 5,514,555, entitled: “Assays and therapeutic methods based on lymphocyte chemoattractants,” issued May 7, 1996, to Springer, T A, et al.). Other suitable methods will be known to one of ordinary skill in the art and can be employed using only routine experimentation.

In particular gp120 inhibitors can be used to enhance migration of antigen-specific immune cells towards an antigen-expressing target cell. The gp120 inhibitor is administered to a subject in need thereof in an amount effective to enhance migration of the antigen-specific immune cells towards an antigen-expressing target cell.

Other aspects of the invention involve antigen-specific immune cells and antigen-expressing cells. An antigen-specific immune cell is an immune cell that specifically recognizes an antigen. An antigen-expressing cell is a cell that expresses an antigen. Preferably, antigen expression is at the cell surface.

An antigen as used herein is a molecule capable of provoking an immune response. Antigens include but are not limited to cells, cell extracts, proteins, polypeptides, peptides, polysaccharides, polysaccharide conjugates, peptide and non-peptide mimics of polysaccharides and other molecules, small molecules, lipids, glycolipids, carbohydrates, viruses and viral extracts and muticellular organisms such as parasites and allergens. The term antigen broadly includes any type of molecule which is recognized by a host immune system as being foreign. Antigens include but are not limited to cancer antigens, microbial antigens such as a viral antigen, a bacterial antigen, a fungal antigen, and a parasitic antigen, and allergens.

A cancer antigen as used herein is a compound, such as a peptide or protein, associated with a tumor or cancer cell surface and which is capable of provoking an immune response when expressed on the surface of an antigen presenting cell in the context of an MHC molecule. Cancer antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens. Cancer antigens include but are not limited to antigens that are recombinantly expressed, an immunogenic portion of, or a whole tumor or cancer. Such antigens can be isolated or prepared recombinantly or by any other means known in the art.

A microbial antigen as used herein is an antigen of a microorganism and includes but is not limited to viruses, bacteria, parasites, and fungi. Such antigens include the intact microorganism as well as natural isolates and fragments or derivatives thereof and also synthetic compounds which are identical to or similar to natural microorganism antigens and induce an immune response specific for that microorganism. A compound is similar to a natural microorganism antigen if it induces an immune response (humoral and/or cellular) to a natural microorganism antigen. Such antigens are used routinely in the art and are well known to those of ordinary skill in the art.

Examples of viruses that have been found in humans include but are not limited to: Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Both gram negative and gram positive bacteria serve as antigens in vertebrate animals. Such gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Cainpylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Examples of fungi include Cryptococcus neoformans, Histoplasma capsulatum, Coccidioides immitis, Blastomyces dermatitidis, Chlamydia trachomatis, Candida albicans.

Other infectious organisms (i.e., protists) include Plasmodium spp. such as Plasmodium falciparum, Plasmodium malariae, Plasmodium ovale, and Plasmodium vivax and Toxoplasma gondii. Blood-borne and/or tissues parasites include Plasmodium spp., Babesia microti, Babesia divergens, Leishmania tropica, Leishmania spp., Leishmania braziliensis, Leishmania donovani, Trypanosoma gainbiense and Trypanosoma rhodesiense (African sleeping sickness), Trypanosoma cruzi (Chagas' disease), and Toxoplasma gondii.

Other medically relevant microorganisms have been described extensively in the literature, e.g., see C. G. A Thomas, Medical Microbiology, Bailliere Tindall, Great Britain 1983, the entire contents of which is hereby incorporated by reference.

“Non-self” antigens are those antigens on substances entering a subject, or exist in a subject but are detectably different or foreign from the subject's own constituents, whereas “self” antigens are those which, in the healthy subject, are not detectably different or foreign from its own constituents. However, under certain conditions, including in certain disease states, an individual's immune system will identify its own constituents as “non-self,” and initiate an immune response against “self-antigens,” at times causing more damage or discomfort as from, for example, an invading microbe or foreign material, and often producing serious illness in a subject.

The gp120 inhibitor may be a G-alpha-i inhibitor, a kinase inhibitor, or a cAMP agonist, but is not so limited. An example of a G-alpha-I inhibitor is a pertussis toxin or a functional equivalent thereof. The kinase inhibitor may be a phosphatidylinositol 3-kinase (PI3-K) inhibitor such as wortmannin, or a tyrosine kinase inhibitor such as genistein or herbimycin. The cAMP agonist may be a cyclic nucleotide, such as 8-Br-cAMP or functional equivalents thereof. It is to be understood that this list is not intended to be limiting and that the invention intends to capture other species of these agents.

Other kinase inhibitors include, but are not limited to, inhibitors of JAK kinases, Cdc7 kinases, KSS1 kinases, ERK kinases, ab1 kinases, cdk2 kinases, cdc2 kinases, cyclic-GMP-dependent kinases, Ca²⁺/calmodulin-dependent kinases, myosin light chain kinases, TGF-β receptor kinases, Mos kinases, Raf kinases, Lck kinases, Src kinases, EGF receptor kinases, PDGF receptor kinases, Weel kinases, tyrosine kinases, cyclic AMP-dependent kinases, protein kinase C, adenosine kinases, as well as other kinase inhibitors. Some specific examples of kinase inhibitors include STI571 (Gleevec™), N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-1 0-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, STI157, CEP2563, 4-(3-chlorophenylamino)-5,6-dimethyl-7H-pyrrolo [2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, EMD121974, H7, staurosporine, SP-203580, PD98059, isoflavones such as genistein, CGP 41251, flavopiridol, p21, cip1, olomoucine, p27kip1, staurosporin, MLCK inhibitors, iodotubercidin, H7, staurosporine, genistein, SP-203580, PD98059, and indolocarbazoles.

The gp120 inhibitor may also be an anti-gp120 antibody or an anti-chemokine receptor antibody, such as an anti-CXCR4 antibody or an anti-CXCR5 antibody. Antibody fragments are also embraced by the invention. Antibodies include polyclonal and monoclonal antibodies, prepared according to conventional methodology.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205.

Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Fully human monoclonal antibodies also can be prepared by immunizing mice transgenic for large portions of human immunoglobulin heavy and light chain loci. See, e.g., U.S. Pat. Nos. 5,545,806, 6,150,584, and references cited therein. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

In some important embodiments, the antigen-specific immune cells are directed towards HIV. Accordingly, the antigen-specific target cell may be a virus-infected cell such as an HIV-infected cell, or it may be a cell free viral component such as cell free HIV virus. In these embodiments, the subject may be one either having an HIV infection or one that is at risk of developing such an infection. An example of a subject at risk of developing an HIV infection is one who has been exposed to HIV, but has not yet manifested symptoms of HIV infection. Other examples of subjects at risk of developing an HIV infection include intravenous drug users, subjects engaged in sexual activity without condoms, and the like. The art is familiar with those subjects who would be considered at risk. The diagnosis of subjects having an HIV infection is routinely carried out by medical professionals and thus is known in the art.

These subjects may be further administered an anti-HIV therapy. As used herein, the terms “anti-HIV therapy” and “anti-HIV agent” are used interchangeably. An “anti-HIV therapy”, as used herein is any therapeutic that is useful for reducing viral load, preventing viral infection, prolonging the asymptotic phase of HIV infection, or prolonging the life of a subject infected with HIV. The anti-HIV therapy may be an inhibitor of HIV replication, such as a protease inhibitor, e.g., HAART, but it is not so limited. In another embodiment the anti-HIV therapy is a cytokine or a chemokine. The cytokine may optionally be a T-cell activating cytokine, such as IL-2. The chemokine may be RANTES or MIP-1α.

The invention further provides methods of screening and identifying pharmacological agents or lead compounds for agents active in modulating the biological activities described herein. One example of such a biological activity is gp120-mediated fugetaxis.

A wide variety of assays for pharmacological agents are provided, including labeled in vitro protein-protein binding assays, electrophoretic mobility shift assays, immunoassays, cell-based assays such as two- or three-hybrid screens, expression assays, etc. For example, three-hybrid screens are used to rapidly examine the effect of transfected nucleic acids on the activity of gp120 fragments. The transfected nucleic acids can encode, for example, combinatorial peptide libraries or antisense molecules. Convenient reagents for such assays, e.g., GAL4 fusion proteins, are known in the art.

Typically, a plurality of assay mixtures are run in parallel with different agent concentrations to obtain a different response to the various concentrations. Typically, one of these concentrations serves as a negative control, i.e., at zero concentration of agent or at a concentration of agent below the limits of assay detection. Candidate agents encompass numerous chemical classes, although typically they are organic compounds. Preferably, the candidate pharmacological agents are small organic compounds, i.e., those having a molecular weight of more than 50 yet less than about 2500, preferably less than about 1000 and, more preferably, less than about 500. Candidate agents comprise functional chemical groups necessary for structural interactions with polypeptides and/or nucleic acids, and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid, the agent typically is a DNA or RNA molecule, although modified nucleic acids as defined herein are also contemplated.

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, acidification, etc. to produce structural analogs of the agents.

Thus, the invention involves polypeptides of numerous size and type that bind specifically to gp120 polypeptides. These polypeptides may act as “masks” in that they mask the fugetactic effect of gp120. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention, including human antibodies. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to the gp120 polypeptide. This process can be repeated through several cycles of reselection of phage that bind to the gp120 polypeptide. Repeated rounds lead to enrichment of phage bearing particular sequences. DNA sequence analysis can be conducted to identify the sequences of the expressed polypeptides. The minimal linear portion of the sequence that binds to the gp120 polypeptide can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the gp120 polypeptides. Thus, the gp120 polypeptides of the invention, or a fragment thereof, can be used to screen peptide libraries, including phage display libraries, to identify and select peptide binding partners of the gp120 polypeptides of the invention. Such molecules can be used, as described, for screening assays, for purification protocols, for interfering directly with the functioning of gp120 polypeptide and for other purposes that will be apparent to those of ordinary skill in the art.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents.

The therapeutics of the invention can be administered by any conventional route, including injection or by gradual infusion over time. The administration may, for example, be oral, intravenous, intraperitoneal, intramuscular, intracavity, subcutaneous, or transdermal. When antibodies are used therapeutically, a preferred route of administration is by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing antibody aerosols without resort to undue experimentation. When using antisense preparations of the invention, slow intravenous administration is preferred.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

In other embodiments, the subject may be further administered an anti-inflammatory agent. An anti-inflammatory agent is an agent that reduces or inhibits altogether an inflammatory response in vivo. Examples of anti-inflammatory agents include but are not limited to Piroxicam, Mefenamic acid, Nabumetone, Sulindac, Tolmetin, Ketorolac, Rofecoxib, Diclofenac, Naproxen, Flurbiprofen, Celecoxib, Oxaprozin, Diflunisal, Etodolac, Fenoprofen, Ibuprofen, Indomethacin, Ketoprofen, Etodolac, Meloxicam, Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rinexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

The invention further provides compositions comprising gp120 molecules and other therapeutic agents such as anti-inflammatory agents, asthma medicaments, and/or allergy medicaments, as recited above.

The preparations of the invention are administered in effective amounts. An effective amount is that amount of a pharmaceutical preparation that alone, or together with further doses, produces the desired response. In the case of treating a condition characterized by an excessive or inappropriate immune response, such as an autoimmune disease, the desired response is inhibiting the excessive or inappropriate immune response. This may involve only slowing the progression or manifestation of the disease temporarily, although more preferably, it involves halting the progression or manifestation of the disease permanently. This can be monitored by routine methods or can be monitored according to diagnostic methods of the invention discussed herein. The effective amounts can be administered in vivo, ex vivo to cells isolated from a subject, or in vitro for diagnostic, research and testing purposes.

Therapeutically effective amounts can also be determined in animal studies. For instance, the effective amount of an agent that inhibits gp120 mediated fugetaxis and/or an anti-HIV therapy to induce a therapeutic response can be assessed using in vivo assays of viral load. Relevant animal models include primates infected with simian immunodeficiency virus (SIV). Generally, a range of doses are administered to the animal along, possibly with a range of anti-HIV therapy doses. Reduction in viral load in the animals following the administration of the active agents is indicative of the ability to reduce the viral load and thus treat HIV infection.

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 0.1 μg to 20 mg/kg/day, more typically from about 1 to 10 mg/kg/day, and most typically from about 1 to 5 mg/kg/day.

Other delivery systems can include time-release, delayed release or sustained release delivery systems. Such systems can avoid repeated administrations of the fugetactic agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) difusional systems in which an active component permeates at a controlled rate from a polymer such as described in U.S. Pat. Nos. 3,832,253, and 3,854,480.

A preferred delivery system of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vessels (LUV), which range in size from 0.2-4.0 μm can encapsulate large macromolecules. RNA, DNA, and intact virions can be encapsulated within the aqueous interior and be delivered to cells in a biologically active form (Fraley, et al., Trends Biochem. Sci., (1981) 6:77). In order for a liposome to be an efficient gene transfer vector, one or more of the following characteristics should be present: (1) encapsulation of the gene of interest at high efficiency with retention of biological activity; (2) preferential and substantial binding to a target cell in comparison to non-target cells; (3) delivery of the aqueous contents of the vesicle to the target cell cytoplasm at high efficiency; and (4) accurate and effective expression of genetic information.

Liposomes may be targeted to a particular tissue by coupling the liposome to a specific ligand such as a monoclonal antibody, sugar, glycolipid, or protein. Liposomes are commercially available from Gibco BRL, for example, as LIPOFECTIN™ and LIPOFECTACE™, which are formed of cationic lipids such as N-[1-(2,3 dioleyloxy)-propyl]-N,N, N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, (1985) 3:235-241.

In one important embodiment, the preferred vehicle is a biocompatible microparticle or implant that is suitable for implantation into the mammalian recipient. Exemplary bioerodible implants that are useful in accordance with this method are described in PCT International application no. PCT/US/03307 (Publication No. WO 95/24929, entitled “polymeric Gene Delivery System”). PCT/US/0307 describes a biocompatible, preferably biodegradable polymeric matrix for containing an exogenous gene under the control of an appropriate promoter. The polymeric matrix is used to achieve sustained release of the exogenous gene in the patient. In accordance with the instant invention, the fugetactic agents described herein are encapsulated or dispersed within the biocompatible, preferably biodegradable polymeric matrix disclosed in PCT/US/03307.

The polymeric matrix preferably is in the form of a microparticle such as a microsphere (wherein an agent is dispersed throughout a solid polymeric matrix) or a microcapsule (wherein an agent is stored in the core of a polymeric shell). Other forms of the polymeric matrix for containing an agent include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used. Preferably when an aerosol route is used the polymeric matrix and fugetactic agent are encompassed in a surfactant vehicle. The polymeric matrix composition can be selected to have both favorable degradation rates and also to be formed of a material which is bioadhesive, to further increase the effectiveness of transfer. The matrix composition also can be selected not to degrade, but rather, to release by diffusion over an extended period of time.

In another important embodiment the delivery system is a biocompatible microsphere that is suitable for local, site-specific delivery. Such microspheres are disclosed in Chickering et al., Biotech. And Bioeng., (1996) 52:96-101 and Mathiowitz et al., Nature, (1997) 386:410-414.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, fugetactic agents are delivered using a bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Examples of non-biodegradable polymers include ethylene vinyl acetate, poly(meth)acrylic acid, polyamides, copolymers and mixtures thereof.

Bioadhesive polymers of particular interest include bioerodible hydrogels described by H. S. Sawhney, C. P. Pathak and J. A. Hubell in Macromolecules, (1993) 26:581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

In addition, important embodiments of the invention include pump-based hardware delivery systems, some of which are adapted for implantation. Such implantable pumps include controlled-release microchips. A preferred controlled-release microchip is described in Santini, J T Jr., et al., Nature, 1999, 397:335-338, the contents of which are expressly incorporated herein by reference.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

In certain embodiments, the agents of the invention are delivered directly to the site at which there is inflammation, e.g., the joints in the case of a subject with rheumatoid arthritis, the blood vessels of an atherosclerotic organ, etc. For example, this can be accomplished by attaching an agent (nucleic acid or polypeptide) to the surface of a balloon catheter; inserting the catheter into the subject until the balloon portion is located at the site of inflammation, e.g. an atherosclerotic vessel, and inflating the balloon to contact the balloon surface with the vessel wall at the site of the occlusion. In this manner, the compositions can be targeted locally to particular inflammatory sites to modulate immune cell migration to these sites. In another example the local administration involves an implantable pump to the site in need of such treatment. Preferred pumps are as described above. In a further example, when the treatment of an abscess is involved, the fugetactic agent may be delivered topically, e.g., in an ointment/dermal formulation. Optionally, the agents are delivered in combination with other therapeutic agents (e.g., anti-inflammatory agents, immunosuppressant agents, etc.).

A better understanding of the present invention and of its many advantages will be had from the following examples, which further describe the present invention and given by way of illustration. The examples that follow are not to be construed as limiting the scope of the invention in any manner. In light of the present disclosure, numerous embodiments within the scope of the claims will be apparent to those of ordinary skill in the art.

### EXAMPLES

The migratory responses of HIV-specific CTLs to varying concentrations of recombinant gp120 were assessed using in vitro and in vivo transmigration assay systems. HIV-specific CTL clones were tested by chromium release assays and flow cytometry for their ability to kill target cells expressing gp120. Altering cell density and employing flat bottom plates in the cytotoxicity assays allowed for the evaluation of cell migration on killing efficacy. Time-lapse video microscopy was used to confirm quantitative results.

CTL clones from HIV-1-infected individuals: HIV-1 specific CTL clones were obtained by cloning stimulated peripheral blood mononuclear cells (PBMC) from HIV-1 infected individuals at limiting dilution, and were characterized for specificity and HLA restriction as previously described (Walker, et al., 1989, PNAS 86:9514-9518; Yang, et al., 1997, 3 Virol 71:3120-3128). CTL clones designated, DMD, ND-25 and ASB-C11 were all HLA B8-restricted CTL clones isolated from different donors, specific for the HIV-1 Nef epitope FL8 (amino acids [aa] 90-97; FLKEKGGL). The CTL clone MHC B60-restricted clone 161JD27 recognized a Gag epitope IL10 (aa 92-101; IEIKDTKEAL). Amino acids are numbered according to the most recent clade B consensus sequence. All cells were free of Mycoplasma as determined by testing with the Mycoplasma tissue culture RNA detection kit (Jen-Probe, San Diego, Calif.).

Cytotoxicity assays: HLA-matched B-lymphoblastoid cell lines (BLCL) were pulsed with the appropriate peptide, incubated with ⁵¹Cr, washed and distributed in either round or flat bottom 96-well plates at varying cell concentrations (Walker, et al., 1987, Nature, 328:345-8). HIV-1-specific CTL were used as effectors in triplicate wells at effector to target (E:T) ratios from 1:1 to 10:1. Cells were incubated for four hours at 37° C. at which point 30 μl of supernatant was harvested. Twelve hours later, gamma counts were measured on a Microplate reader (Packard Instrument Company, Downers Grove, Ill.). The percentage of specific cytotoxicity was calculated as follows: percentage specific lysis=[(experimental release−spontaneous release)/(maximum release−spontaneous release)]×100. The average spontaneous release of ⁵¹Cr from target cells was always <20% of maximum release.

Mononuclear cell preparation and sorting of subpopulations of T-cells: Peripheral blood was obtained from healthy adult donors according to a protocol approved by the Institutional Review Board. Ficoll-Hypaque (Pharmacia Biotech Inc., Piscataway, N.J.) density-gradient centrifugation was used to isolate peripheral blood mononuclear cells. Cells were then stained with saturating amounts of phycoerythrin-conjugated anti-CD4 or -CD8 mAb and fluorescein isothyiocyanate-conjugated anti-CD45RA or -CD45RO (Becton Dickinson, San Jose, Calif.). The desired subpopulations of peripheral blood cells were sorted using a FACS Vantage sorter (Becton Dickinson) and cultured overnight in Iscove's modified medium containing 0.5% fetal calf serum (Life Technologies, Carlsbad, Calif.) before their use in transmigration assays. The purity of each T-cell subpopulation was determined to be greater than 99% by immunophenotyping.

Transmigration assays: Transmigration assays were performed in a transwell system with a polycarbonate membrane of 6.5-mm diameter with 5 μm pore size (Corning, Corning, N.Y.) as described (Kim et al., 1998, Blood 91:4434-4443; Poznansky et al., 2000, Nat Med 6:543-548). Purified T-cell subpopulations (5×10⁴ cells) were added to the upper chamber of each well in a total volume of 150 μl Iscove's modified media. SDF-1α (PeproTech, Rocky Hill, N.J.) or recombinant HIV-1 gp120 (Immunodiagnostics, Woburn, Mass., AIDS Reagent Repository, NIH or RW) was used at concentrations ranging from 2 ng/ml to 2 g/ml in the lower, upper, or both lower and upper chambers of the transwell to generate a standard “checkerboard” analysis matrix of positive, negative and absent gradients. Recombinant variable loop deletion mutants of HIV-1_{IIIB} gp120 including V1, V1.V2, and V1.V2.V3, were also used in these assays. Transwells were incubated for 3 hours at 37° C., after which cells were collected from the lower chamber and counted.

Transduction of target cell lines: Recombinant adeno-associated virus (rAAV) vectors were used to deliver HIV-1_{HXB2} env, or a control gene, red fluorescent protein (RFP), into target cells. Mock transduction was performed as an additional control. BLCL were washed in RPMI and 10⁶ cells per well aliquoted in minimal volume in 24-well tissue culture plates. Cells were incubated for 90 minutes with 50 μl of rAAV (MOI 2-4), after which 0.5 ml of RPMI with 20% fetal calf serum was added to each well. Successful transduction was confirmed after 48 hours with indirect cytofluorometry for cell surface expression of envelope glycoproteins in the case of env or fluorescent microscopy in the case of RFP, and cells were used immediately as targets in cytotoxicity assays. Secretion of HIV-1gp120 was confirmed by performing an ELISA (Immunodiagnostics, Woburn, Mass.) on culture supernatants from AAV transduced cells. The antibodies used in the experiments were obtained through the AIDS Research and Reference Reagent Program from M. Rosner.

Immunization and challenge of mice: C57/BL6 mice and OT-1 mice (Jackson Laboratories, Bar Harbor, Me.) were immunized subcutaneously against chicken ovalbumin (Ova) (Sigma) and subsequently challenged with a second intraperitoneal (IP) injection of Ova as previously described (Poznansky et al., 2000, Nat Med 6:543-548). Twenty-four hours after IP Ova challenge, experimental mice received a second IP injection containing low (20 ng/ml) or high (200 ng/ml) dose HIV-1_{IIIB} gp120. Recombinant HIV-1_{IIIB} gp120 containing deletions of the V1.V2 and V1.V2.V3 loops were also tested at high and low doses. Control mice were exposed to IP injections of N-saline or boiled gp120. Mice were euthanized, 3 and 24 hours after the second IP injection and peritoneal lavage with 5 ml of PBS was performed. Total number of viable nucleated cells per ml of peritoneal fluid was determined with a hemocytometer and by trypan blue exclusion. Peritoneal fluid obtained in this way contained less than 0.1% red blood cells. Flow cytometry was performed on peritoneal fluid cells using fluorochrome-conjugated antibodies against mouse T cells (phycoerythrin-anti CD3, biotin-anti CD8 and APC-anti CD4) (all from Caltag Laboratories). Second-step staining of biotin-conjugated antibodies used streptavidin-peridinnin chlorophyll protein (Becton Dickinson). The proportion of T cells of each subpopulation was determined as a percentage of the total nucleated cell fraction in the peritoneal fluid.

Statistical Analysis: All experiments were performed at least in triplicate, with the data shown representative of all results. The data were analyzed for statistical significance using the Wilcoxon signed rank exact test or a two-tailed Student's paired T-test.

### Results

CXCR-4-specific recombinant X4 gp120 elicited a migratory response of T-cells including HIV-specific CTL movement away from the recombinant HIV protein. Migration away from gp120 was concentration dependent, CD4-independent and was inhibited by anti-CXCR-4, pertussis toxin and 8-Br-cAMP. Recombinant gp120 was also shown to be active in vivo in significantly reducing T-cell infiltration at a site of antigen challenge. It was also demonstrated that the active movement of HIV-specific CTL clones was essential for their ability to kill target cells with decreased cell lysis seen in response to lower cell density, despite maintenance of equal effector:target ratios. Time-lapse video microscopy allowed for qualitative confirmation of the CTL/target cell interaction at various cell densities.

SDF-1, the natural ligand of CXCR4, serves as a bidirectional cue for T-cells—attracting at one concentration and repelling at a higher concentration via a CXCR4-dependent and pertussis toxin sensitive mechanism (Poznansky et al., 2000, Nat Med 6:543-548). A similar finding for X4 gp120 had been postulated. Mature resting CD8+CD45RO+ T-cells isolated from the peripheral blood of healthy volunteers were used in transmigration assays to quantitate their migratory responses to positive, negative and absent gradients of recombinant HIV-1_{IIIB} gp120. Standard checkerboard analyses of human T-cell migration demonstrated that gp120 could serve as a bidirectional cue for subpopulations of human resting peripheral blood CD8+ T-cells (Table 1).

TABLE 1

| Top Chamber | | | | | | |
|---|---|---|---|---|---|---|
| [gp120][gp120] | | | | | | |
| Lower Chamber | | 0 ng/ml | 2 ng/ml | 20 ng/ml | 200 ng/ml | 2 μg/ml |
| 0 | ng/ml | 4.5 +/− 0.9% | 4.8 +/− 0.6% | 5.9 +/− 0.5% | 16.1 +/− 1.2% | 7.6 +/− 0.7% |
| 2 | ng/ml | 5.1 +/− 0.7% | 4.6 +/− 0.8% | 3.4 +/− 0.6% | 10.1 +/− 1.1% | 11.2 +/− 1.8% |
| 20 | ng/ml | 13.6 +/− 1.5% | 5.8 +/− 1.3% | 2.8 +/− 0.5% | 7.2 +/− 1.3% | 8.6 +/− 0.5% |
| 200 | ng/ml | 6.0 +/− 0.7% | 8.9 +/− 0.9% | 6.6 +/− 1.2% | 3.7 +/− 0.5% | 5.7 +/− 1.1% |
| 2 | μg/ml | 5.1 +/− 1.0% | 11.6 +/− 1.4% | 7.1 +/− 0.9% | 6.3 +/− 0.8% | 4.6 +/− 0.7% |

Table 1 depicts checkerboard transmigration analysis of CD8+CD45RO+ T-cells in response to recombinant HIV-1_{IIIB} gp120. Approximately 10⁵ cells were placed in the upper chamber and X4 gp120 was added at the indicated concentrations to the upper and/or lower chamber creating a negative gradient (above the diagonal), positive gradient (below the diagonal) or equal concentrations in both chambers (along the diagonal) of HIV-1 gp120.

At a concentration of 20 ng/ml, HIV-1_{IIIB} gp120 elicited maximal chemotaxis (13.6%+/−1.5%)—movement towards the recombinant protein. In contrast, higher concentrations of HIV-1_{IIIB} gp120 (200 ng/ml) caused maximal migration (16.1%+/−1.2%) of T-cells away from the HIV-1 protein—fugetaxis. Minimal random movement of T-cells, or chemokinesis, was seen in response to HIV-1_{IIIB} gp120 presented in the absence of a gradient. Transmigration experiments were repeated using three different sources of recombinant X4 gp120 and similar T-cell migratory responses from mature T-cell subpopulations were observed (data not shown). It had been concluded that X4 gp120 served as a bidirectional cue for T-cells in vitro, and that movement towards or away from the HIV-1 protein was concentration-dependent.

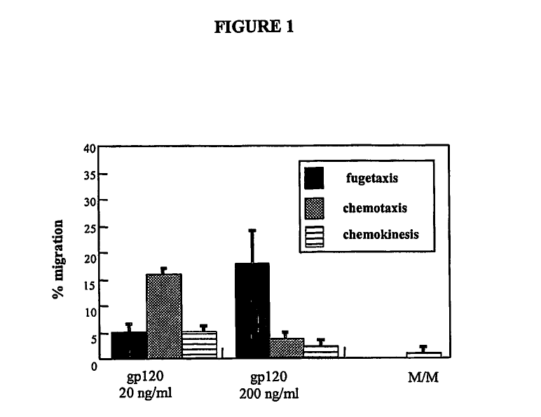

In order to determine whether X4gp120 could also serve as bidirectional cue for HIV-antigen specific CTL migration, the effect of the recombinant HIV gp120 on the migration of CTL clones was examined. Once again, chemotaxis occurred at a peak concentration of 20 ng/ml and maximal migration away from X4 gp120, or fugetaxis, occurred at the higher concentration of 200 ng/ml (FIG. 1). In conclusion, both primary CD8+CD45RO+ cells and HIV-specific CD8+ CTL demonstrate concentration-dependent movement towards and away from the HIV-1 protein, X4 gp120, in vitro.

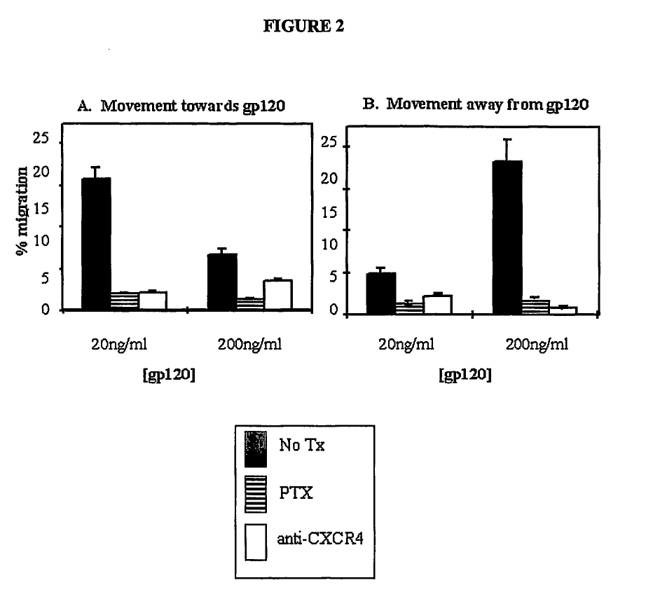

Specific components of the Gᵢ-protein coupled receptor signaling pathway for SDF-1 can be blocked by a number of different chemical inhibitor (Poznansky et al., 2000, Nat Med 6:543-548; Poznansky et al., 2002, Clin. Invest. 109:1101-1110; Sotsios et al., 1999, J. Immunol. 163:5954-5963; Vlahakis et al. 2001, J. Clin. Invest. 107:207-215). The inhibitor profile for movement of resting T-cell subpopulations towards and away from recombinant X4 gp120 in transmigration assays had been established. CD8+CD45RO+ T-cell migration towards and away from HIV-1gp120 was significantly inhibited by the Gᵢ-protein inhibitor pertussis toxin (p=0.0013) and CXCR4-binding antibody 12G5 (p=0.008) suggesting that T-cells migrate both towards and away from X4 HIV-1 gp120 in a manner similar to SDF-1 (FIG. 2).

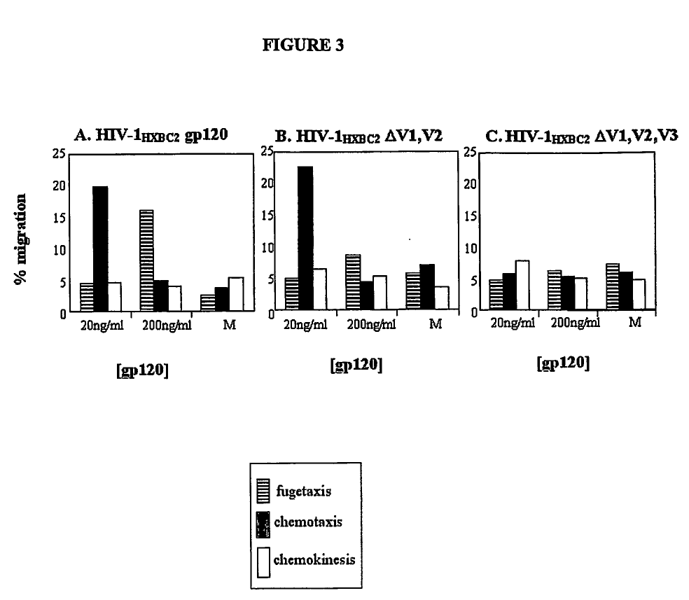

The precise binding site of gp120 to CXCR4 is not yet mapped. However, it has been demonstrated that the V3 loop plays a significant role in this interaction (Rizzuto, et al., 1998, Science 280:1949-1953; Verrier et al., 1999, AIDS Res. Hum. Retroviruses, 15:731-743). Guided by previous studies of the receptor-ligand interaction between HIV-1 gp120 and CXCR4 (Basnaciogullari et al., 2002, J. Virol. 76:10791-10800.) specific deletion mutations of HIV-1_{IIIB} gp120 were used in order to investigate which structural components might play a role in the observed migratory response of CD8+ T-cells. The migratory responses of resting T-cell subpopulations in response to mutants of HIV-1_{IIIB} gp120 containing V1.V2 or V1.V2.V3 loop deletions at concentrations of 20 ng/ml or 200 ng/ml were assessed (FIG. 3). The deletion of the V1 and V2 loops of HIV-1_{IIIB} gp120 led to exclusive movement of T-cells towards gp120 (15%+/−1.1%) and complete loss of the signal to move away from gp120. Deletion of the V1, V2 and V3 loops led to abrogation of movement of resting T-cells both towards and away from gp120. These results suggest that the V3 loop of X4gp120 may play a significant role in signaling CD8+ T-cell migration.

In order to test the hypothesis that migration plays a direct role in CTL efficacy, the ⁵¹Cr release assay (Siliciano, et al., 1988, Cell 54:561) was modified in two ways. First, the cytotoxicity of HIV-specific CTL was quantitated by the standard technique in a round bottom 96-well plate and compared to results of assays performed in a flat bottom 96-well plate (FIG. 4A). Demonstration of significantly decreased lysis (p=0.027) when effectors and targets were incubated in the flat bottom wells (as opposed to being pelleted together in the round bottom wells) support the view that cell movement plays a role in determining CTL efficacy. Video microscopy demonstrated that effector cells incubated with targets in a flat bottom well moved from one target cell to another inducing lysis whereas cells incubated in the round bottom well did not exhibit discernable migration during the incubation period (data not shown). Secondly, the assay was further modified to delineate between percent specific lysis due to the E:T ratio and the percent specific lysis attributable to the total number of cells placed in the flat bottom well. In this modified flat ⁵¹Cr release assay the total number of cells per well was kept constant at each E:T ratio as compared to the standard assay where both the E:T ratio and total number of cells per well decrease. As expected, at the E:T ratio of 10:1, conditions were identical for the standard and modified flat bottom assays (110,000 cells/well) and no differences in percent specific lysis were seen. At the E:T ratios of 5:1 and 1:1, however, CTL killing efficacy differed significantly (p=0.031) between the two conditions (FIG. 4B). These data suggest that the total number of cells per well is an important variable when the ⁵¹Cr release assay is performed in a flat bottom well.

The probability theory was used to mathematically model the spatial relationship between target and effector cells in a flat bottom well and calculate the distance a CTL has to migrate to reach a target cell for a given number of cells per well (Stoyan et al., 1995, Stochastic geomtery and its applications, 2nd edition, John Wiley & Sons, New York; Stoyan, et al., 1994, Fractals, random shapes, and point fields, John Wiley & Sons, New York). The model assumes a random distribution of both effector and target cells on the surface of the flat-bottom well, and that the statistics governing the position of one cell type is not influenced by the other. Under these assumptions, the expected distance (D) between a CTL and a target cell equals a universal, dimensionless constant (K) divided by the square root of the density of the target cells in the flat bottom well, (ₜ) (Equation 1). The density of target cells equals the number of targets placed in the well (n) divided by the area of the well. In this case, the well is a circle, hence Equation 2. Experimentally, a highly significant positive correlation between observed CTL lysis and calculated distance required to reach their targets at all E:T ratios tested had been found (FIG. 4C). These data support the concept of a relationship between CTL efficacy and their ability to actively migrate to target cells, and also provides a model system in which to examine the impact of molecules which effect cell migration on CTL efficacy.

$$\begin{matrix}D = \frac{K}{\sqrt{{λ}_{t}}} = 1 / 2 \sqrt{{λ}_{t}} & Equation 1 \\ {λ}_{t} = \frac{{n}_{( targets )}}{{π ( {r}_{well} )}^{2}} . & Equation 2\end{matrix}$$

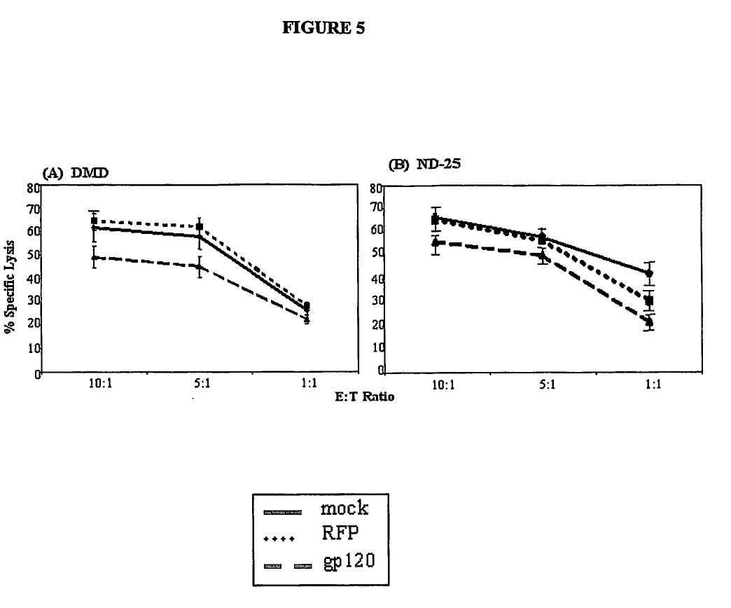

Using modified ⁵¹Cr release assay described above, the effect of the expression of X4 gp120 by the target cell on CTL efficacy had been investigated. Autologous BLCL were transduced with recombinant adeno-associated virus (rAAV) vector encoding HIV-1_{HXBc2} env. Control rAAV vector expressed red fluorescent protein (RFP). Cells were used as targets in the modified ⁵¹Cr release assay 48 hours after infection with viral constructs. Mock transduced BLCL provided an additional control. Surface expression and secretion of gp120 by target cells was confirmed by indirect immunofluorescence and supernatant gp120 ELISA, respectively. The target cells transduced with env demonstrated significantly lower percent specific lysis by two nef-specific clones compared to the targets transduced with RFP (p=0.008 for DMD, p=0.0002 for ND-25) or to the mock transduced cells (p=0.02 for DMD, p=0.0004 for ND-25) (FIG. 5). HIV-1 gp120 has been previously reported to mediate CD4+ and CD8+ T-cell apoptosis through its interaction with the CXCR4 receptor (Vlahakis et al. 1987, 328:345-8). The mock ⁵¹Cr release assays without radioisotope labeling were performed and after four hours, the effector and target cells were labelled with APC-anti CD8 (Caltag) and 7-Amino-Actinomycin D (Sigma). Levels of apoptosis were similar between CTL incubated with target cells expressing gp120 versus controls (data not shown). These data support the view that the reduction in CTL efficacy seen when target cells expressed X4 gp120 was not due to increased CTL death. In this way, it had been demonstrated that X4 gp120 expression by target cells reduced lysis by CTL.

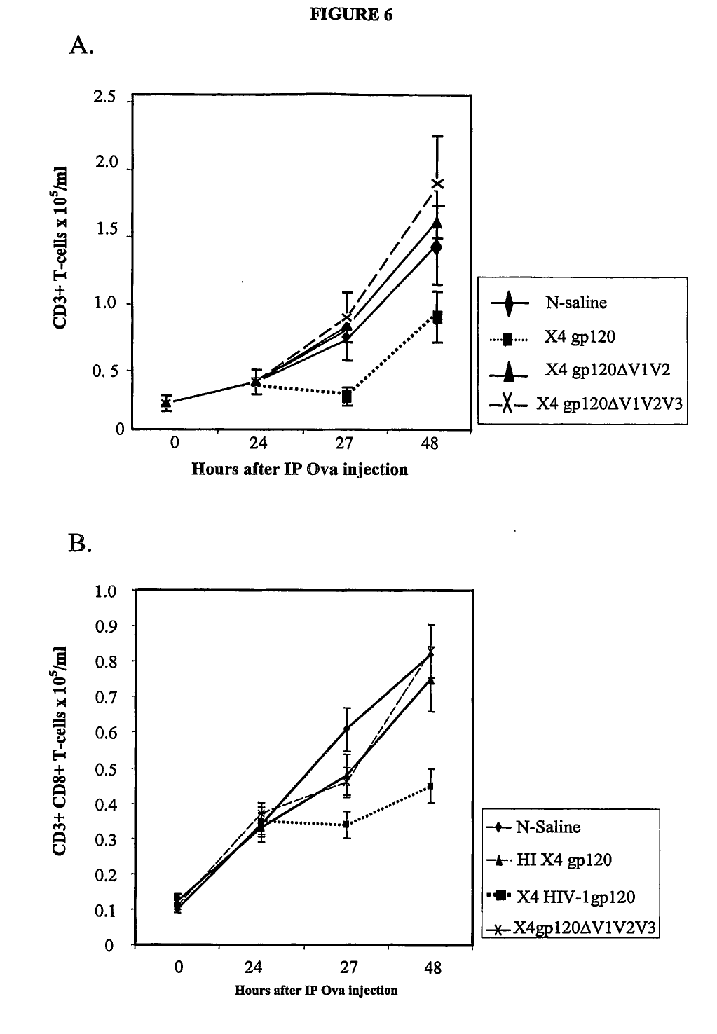

The chemokine receptor for SDF-1 and X4 gp120, CXCR4, is structurally and functionally highly conserved between humans and mice, sharing 91% amino acid sequence homology (Heesen et al., 1996, J Immunol 157:5455-5460). As in humans, X4 gp120 elicits chemotaxis in murine T cells expressing CXCR4 in a CD4-independent manner (Shieh, et al., 1998, J Virol 72:4243-4249). It had been confirmed that migratory responses of resting murine CD8+ T-cells to both human SDF-1 and recombinant X4 gp120 closely resemble those of human resting CD8+ T-cells within transmigration assays (data not shown). It had been previously demonstrated that a concentration of SDF-1 of 126 nM can abrogate established immune responses in a mouse model (Poznansky et al., 2000, Nat Med 6:543-548). Using a similar protocol, it had been examined whether X4 gp120 could do the same. C57 BL/6 mice immunized against chicken Ovalbumin (Ova) were challenged 3 days later with an intraperitoneal (IP) injection of Ova (Time 0). Twenty-four hours later, experimental mice received a second IP injection containing high (200 ng/ml) or low (20 ng/ml) dose X4 gp120. Recombinant loop deleted forms of X4 gp120 were also tested at high and low doses. Control mice were exposed to IP injections of N-saline or boiled recombinant X4 gp120. High dose X4 gp120 led to a significant reversal in T-cell infiltration into the IP cavity in response to antigen to which the mouse had been sensitized (FIG. 6A). Compared to control animals, the mice that received 200 ng/ml X4 gp120 were found to have significantly reduced T-cell infiltration into the peritoneal cavity in response to antigen challenge (p=0.04, Student's t test) 27 hours after initial IP Ova injection (3 hours after the second injection). At 48 hours, the difference had lessened, but decreased CD3+ cells were still seen in the mice receiving X4gp120 versus controls (p=0.05). Recombinant loop mutants of X4gp120 had no detectable effect on the infiltration of immune effector cells into the intraperitoneal cavity. The “chemotactic” concentration of gp120 (20 ng/ml) did not augment T-cell infiltration into the peritoneal cavity beyond the robust reaction seen with antigen stimulation alone (data not shown). These data were similar to those generated with a low “chemotactic” concentration (12.6 nM) of SDF-1 which did not augment T cell infiltration into the peritoneal cavity beyond the levels induced by ovalbumin alone (Poznansky et al. 2000, Nat Med. 6:543-8).

Antigen specific CD8+ T-cell migration was examined in the context of OT-1 mice engineered to express an Ova-specific TCR. We determined the number of CD3+CD8+ T-cells migrating into the intraperitoneal cavity in response to challenge with Ova as described above. Recombinant X4 gp120 led to a significant reduction of CD3+CD8+ T-cell infiltration into the peritoneal cavity as compared to control N-saline administration (p=0.038) or administration of heat-inactivated HIV-1gp120 (p=0.47) or HIV-1gp120 deleted of the V1, V2 and V3 loops (p=0.044) at 48 hours following the intraperitoneal injection of Ova (FIG. 6B).

### CONCLUSIONS

The modified ⁵¹Cr release assay described above assesses CTL efficacy in a way that incorporates the critical factor of effector cell migration necessary to mediate contact-dependent cell lysis in vivo. This assay allows for investigation of CTL migratory and effector capabilities not only in the setting of HIV, but also in the case of other infections where the detectability of CTL does not consistently correlate with viral control (Lee, et al., 1999, Nat Med 5:677-685). It had been shown that high concentrations of the HIV-1 protein, X4 gp120, cause T cells and, in particular, HIV-specific CTL, to actively migrate away from the chemokinetic stimulus in vitro and that the expression of gp120 on target cells reduces CTL efficacy. This novel mechanism of immune evasion may be more broadly applicable to other retroviruses, poxviruses and herpesviruses, all of which have been shown to encode viral proteins which influence cell migration. Furthermore, selective manipulation of chemotactic and fugetactic signals could allow augmentation of the host immune response thereby providing a novel immunotherapeutic strategy and potentially enhancing vaccine efficacy.

It should be understood that the preceding is merely a detailed description of certain embodiments. It therefore should be apparent to those of ordinary skill in the art that various modifications and equivalents can be made without departing from the spirit and scope of the invention, and with no more than routine experimentation. It is intended to encompass all such modifications and equivalents within the scope of the appended claims.

1. A method for inhibiting an abnormal immune response comprising
administering to a subject in need thereof a gp120 molecule or functional equivalent thereof in an amount effective to inhibit an abnormal immune response.

2. The method of claim 1, wherein the abnormal immune response includes undesired infiltration of T cells.

3. The method of claim 2, wherein the gp120 molecule inhibits the undesired infiltration of T cells.

4. The method of claim 1, wherein the abnormal immune response is selected from the group consisting of autoimmune disease, immune hypersensitivity, allergy, asthma, graft-versus-host disease (GVHD), and inflammation.

5. The method of claim 1, wherein the abnormal immune response is reduced to a normal level.

6. The method of claim 1, wherein the gp120 molecule is a gp120 polypeptide or a fragment thereof.

7. The method of claim 1, wherein the gp120 molecule is a gp120 nucleic acid molecule.

8. The method of claim 1, wherein the gp120 molecule is a soluble gp120 molecule or a cell bound gp120 molecule.

9. A method for enhancing migration of antigen-specific immune cells towards an antigen-expressing target comprising administering to a subject in need thereof an agent that inhibits gp120-mediated fugetaxis in an amount effective to enhance migration of antigen-specific immune cells towards an antigen-expressing target.

10. The method of claim 9, wherein the antigen-specific immune cells are antigen-specific cytotoxic T lymphocytes.

11. The method of claim 9, wherein the antigen-specific target is a cell free HIV virus or a cell-associated HIV virus.

12. The method of claim 9, wherein the agent is selected from the group consisting of anti-chemokine receptor antibody or a fragment thereof, a G-alpha-i inhibitor, a kinase inhibitor, and a cAMP agonist.

13. The method of claim 12, wherein the G-alpha-i inhibitor is a pertussis toxin or a functional equivalent thereof.

14. The method of claim 12, wherein the kinase inhibitor is selected from the group consisting of a phosphatidylinositol 3-kinase (PI3-K) inhibitor and a tyrosine kinase inhibitor.

15. The method of claim 14, wherein the phosphatidylinositol 3-kinase (PI3-K) inhibitor is wortmannin.

16. The method of claim 14, wherein the tyrosine kinase inhibitor is genistein or herbimycin.

17. The method of claim 12, wherein the cAMP agonist is a cyclic nucleotide.

18. The method of claim 17, wherein the cyclic nucleotide is 8-Br-cAMP or a functional equivalent thereof.

19. The method of claim 9, wherein the agent is administered systemically or in a sustained release vehicle.

20. The method of claim 11, further comprising administering an anti-HIV agent to the subject.

21. The method of claim 11, wherein the subject has an HIV infection.

22. The method of claim 11, wherein the subject is at risk of developing an HIV infection.

23. The method of claim 22, wherein the subject has been exposed to HIV.

24. The method of claim 9, wherein the antigen-specific immune cells with cytotoxic activity are cytotoxic CD8+ T lymphocytes, natural killer (NK) cells, neutrophils, cytotoxic CD4+ T lymphocytes, and macrophages.

25. The method of claim 9, wherein the anti-chemokine receptor antibody or a fragment thereof is an anti-CXCR4 antibody or a fragment thereof, or anti-CXCR5 antibody or a fragment thereof.

26. The method of claim 9, wherein the antigen-specific immune cell is an antigen-specific immune cell with cytotoxic activity.

27. A method for measuring activity of immune cells with cytotoxic activity comprising
placing at least one effector cell and at least one target cell in a flat bottom chamber,
incubating the cells for a time sufficient to allow lysing of the at least one target cell by the at least one effector cell, and
determining a proportion of target cells lysed,
wherein the proportion of target cells lysed is measured using a non-fluorescent assay, and
wherein the effector cell is an immune cell with cytotoxic activity.

28. The method of claim 27, wherein the non-fluorescent assay is radioactivity release.

29. The method of claim 27, wherein the at least one effector cell and the at least one target cell are present in a pre-defined ratio.

30. The method of claim 29, wherein the pre-defined ratio is selected from the group consisting of 1000:1, 750:1, 500:1, 250:1, 100:1, 50:1, 10:1, 5:1 and 1:1.

31. The method of claim 27, further comprising comparing results of the assay to a standard curve.

32. The method of claim 27, wherein a total number of cells per flat bottom chamber is constant.

33. The method of claim 27, wherein the total number of cells per flat bottom chamber is selected from the group consisting of at least 10,000, at least 20,000, at least 25,000, at least 50,000, at least 75,000, at least 100,000, at least 125,000, at least 150,000, at least 175,000, and at least 200,000.

34. The method of claim 27, wherein the immune cell with cytotoxic activity is selected from the group of cells consisting of cytotoxic CD8+ T lymphocytes, natural killer (NK) cells, neutrophils, cytotoxic CD4+ T lymphocytes, and macrophages.

35. The method of claim 27, wherein the immune cell with cytotoxic activity is a cytotoxic CD8+ T lymphocyte.

36. A method for measuring activity of immune cells with cytotoxic activity comprising
placing at least one effector cell and at least one target cell in a flat bottom chamber,
incubating the cells for a time sufficient to allow lysing of the at least one target cell by the at least one effector cell, and
determining a proportion of target cells lysed,
wherein the proportion of target cells lysed is measured using a flow cytometer or a radioactivity counter, and
wherein the effector cell is an immune cell with cytotoxic activity.

37. The method of claim 36, wherein the radioactivity counter is used to measure radioactive chromium release.

38. The method of claim 36, wherein the flow cytometer is used to measure propidium iodide, 7-AAD or fluorogenic caspase substrate.

38. The method of claim 36, wherein the at least one effector cell and the at least one target cell are present in a pre-defined ratio.

40. The method of claim 38, wherein the pre-defined ratio is selected from the group consisting of 1000:1, 750:1, 500:1, 250:1, 100:1, 50:1, 10:1, 5:1 and 1:1.

41. The method of claim 36, further comprising comparing results of the assay to a standard curve.

42. The method of claim 36, wherein a total number of cells per flat bottom chamber is constant.

43. The method of claim 36, wherein the total number of cells per flat bottom chamber is selected from the group consisting of at least 10,000, at least 20,000, at least 25,000, at least 50,000, at least 75,000, at least 100,000, at least 125,000, at least 150,000, at least 175,000, and at least 200,000.

44. The method of claim 36, wherein the immune cell with cytotoxic activity is selected from the group of cells consisting of cytotoxic CD8+ T lymphocytes, natural killer (NK) cells, neutrophils, cytotoxic CD4+ T lymphocytes, and macrophages.

45. The method of claim 36, wherein the immune cell with cytotoxic activity is a cytotoxic CD8+ T lymphocyte.

46. A method for measuring activity of immune cells with cytotoxic activity comprising
placing at least one effector cell and at least one target cell in a flat bottom chamber,
incubating the cells for a time sufficient to allow lysing of the at least one target cell by the at least one effector cell,
determining a proportion of target cells lysed, and
comparing the proportion of target cells lysed to a standard curve,
wherein the effector cell is an immune cell with cytotoxic activity.

47. The method of claim 46, wherein the proportion of target cells lysed is measured by fluorescence or radioactivity release.

48. The method of claim 46, wherein the proportion of target cells lysed is measured using a flow cytometer or a radioactivity counter.

49. The method of claim 48, wherein the radioactivity counter is used to measure radioactive chromium release.

50. The method of claim 46, wherein the flow cytometer is used to measure propidium iodide, 7-AAD or fluorogenic caspase substrate.

51. The method of claim 46, wherein the at least one effector cell and the at least one target cell are present in a pre-defined ratio.

52. The method of claim 51, wherein the pre-defined ratio is selected from the group consisting of 1000:1, 750:1, 500:1, 250:1, 100:1, 50:1, 10:1, 5:1 and 1:1.

53. The method of claim 46, wherein the total number of cells per flat bottom chamber is constant.

54. The method of claim 46, wherein the total number of cells per flat bottom chamber is selected from the group consisting of at least 10,000, at least 20,000, at least 25,000, at least 50,000, at least 75,000, at least 100,000, at least 125,000, at least 150,000, at least 175,000, and at least 200,000.

55. The method of claim 46, wherein the immune cell with cytotoxic activity is selected from the group of cells consisting of cytotoxic CD8+ T lymphocytes, natural killer (NK) cells, neutrophils, cytotoxic CD4+ T lymphocytes, and macrophages.

56. The method of claim 46, wherein the immune cell with cytotoxic activity is a cytotoxic CD8+ T lymphocyte.


SEQUENCE LISTING (7 sequences)

SEQ ID NO: 1 - protein, 11 aa, Unknown Organism
  Description of Unknown Organism Substance P peptide
RPKPQQFFGLM

SEQ ID NO: 2 - protein, 8 aa, Human immunodeficiency virus type 1
FLKEKGGL

SEQ ID NO: 3 - protein, 10 aa, Human immunodeficiency virus type 1
IEIKDTKEAL

SEQ ID NO: 4 - DNA, 9201 bp, Human immunodeficiency virus type 1
cagatctgagcctgggagctctctggctagcggaggaacccactgcttaagcctcaataaagcttgccttgagtgcttcaagtagtgtgtgcccgtctgattgtgtgactctggtaactagagatccctcagaccactctagactgtgtaaaaatctctagcagtggcgcccgaacaggacttgacggtaatagggactcgaaagcgaaagttccagagaagatctctcgacgcaaggactcggcttgctgaggtgcgcacagcaagaggcgagagcggcgactggtgagtacgccaaattttgactagcggaggctagaaggagagagatgggtgcgagagcgtcagtattaagtgggggaaaattagatgcatgggagaaaattcggttaaggccagggggaaagaaaaaatatagactaaaacatctagtatgggcaagcagggagctggaaagattcgcacttaaccctggccttttagaaacagcagagggatgtcaacaactaatggaacagttacaatcagctctcaggacaggatcagaagaacttaaatcattatttaatacaatagcaaccctttggtgcgtgcatcaaaggatagacataaaagacaccaaggaagccttagataaaatggaggaaatacaaaataagagcaagcaaaaggtacagcagacagcagctgccacaggaagcagcagccaaaattaccctatagtgcaaaatgccaaagggcaaatgacacatcagtccatgtcacctagaactttaaatgcatgggtgaaagtaatagaagaaaagggtttcagcccagaagtaatacccatgttttcagcattatcagagggagccaccccgcaagatttaaatatgatgctaaacatagtggggggacaccaggcagcaatgcagatgttaaaagataccatcaatgaggaagctgcagaatgggatagggtacatccagtacatgcagggcctattccaccaggccagatgagggaaccaaggggaagtgacatagcaggaactactagtacccttcaagaacaaataggatggatgacaagcaatccacctatcccagtgggagaaatctataaaagatggatagtcctgggattaaataaaatagtaaggatgtatagccctgtcagcattttggacataagacaagggccaaaagaaccctttagagattatgtagataggttctttaaaactttgagagctgaacaagctacgcaggaggtaaaaaactggatgacagaaaccttgctggtccaaaatgcgaatccagactgtaagtccattttaagagcattaggaacaggggctacattagaagaaatgatgacagcatgtcagggagtgggaggacctggccataaagcaagggttttggctgaggcaatgagtcaagtacaacaggccaacgtaatgatgcagagaggcaattttaggggccagagaacaataaagtgtttcaactgtggcaaagaaggacacctagccagaaattgcaaggcccctaggaaaaggggctgttggaaatgtgggaaagaaggacaccaaatgaaagactgtactgagagacaggctaattttttagggaaaatttggccttccagcaaggggaggccaggaaattttcctcagagcagaccggaaccaacagctccaccagcagagagctttgggatgggggaagagatacctccctctccacagcaggaaccgagggacaaggggctatatcctcctttaacttccctcaaatcactctttggcaacgacccttagttacagtaagaatagagggacagctaatagaagccctattagacacaggagcagatgatacagtattagaagacataaatttaccaggaaaatggaaaccaaaaatgatagggggaattggaggttttatcaaagtaagacaatatgatcaaatacttatagaaatttgtggaaaaaaggccataggtacagtattagtaggacctacacctgtcaacataattggacgaaatatgttgactcagattggttgtactttaaattttccaattagtcctattgaaactgtgccagtaaaattaaagccaggaatggatggcccaaaggttaaacaatggccattgacagaagaaaaaataaaagcattaacagacatttgtacagagatggaaaaggaaggaaaaatttcaaaaattggacctgaaaatccatacaatactccagtatttgccataaagaaaaaggatagtactaaatggagaaaattagtagatttcagagaactcaataagagaactcaagatttctgggaggtccagttaggaatacctcatcccgcgggattaaaaaagaaaaaatcagtaacagtgctagatgtgggggatgcatatttttcagttcccttagataaagactttagaaagtatactgcatttactatacctagtgtaaataatgagacaccagggattagataccagtacaatgtgcttccacagggatggaaagggtcaccggcaatatttcaggcaagcatgacaaaaatcttagagccctttagaacaaaaaatccagagatagtgatctaccaatacatggatgatttatatgtaggatctgatttagaaatagggcagcatagagcaaaaatagaggagttgagaggacatctactgaaatggggatttaccacaccagacaaaaagcatcagaaagaacctccatttctttggatgggttatgaactccatcctgataaatggacagtccagcctgtagaactaccagaaaaagacagctggactgtcaatgatatacagaaattagtgggaaaactaaattgggcaagtcagatttatgcaggaattaaaataaagcaactgtgtagactcctcaggggagccaaagcactaacagatatagtagcactgactgaggaagcagaattagaattggcagagaacagggaaattctaaaagaacctgtacatggggtatattatgacccaacaaaagacttagtagcagaattacagaaacaagggcaagaccaatggacatatcaaatttatcaagagccatttaaaaatttaaaaacaggaaaatatgcaaaaaagaggtctgcccacactaatgatgtaaagcaattaacagaggtagtgcaaaaagtagctatggaaagcatagtaatatggggaaagacccctaaatttagactacccatacaaagagagacatgggaaacatggtggatggagtattggcaggctacctggattcctgaatgggagtttgtcaatacccctcctctagtaaaattatggtaccagttagaaaaagaccccatagtaggagcagaaactttctatgtagatggagcagctaatagagagactaagataggaaaagcagggtatgtcactgacagaggaagacaaaaggttgtttccctaactgagacaacaaatcaaaagactgaattacatgcaattcatttagccttacaggattcaggatcagaagtaaatatagtaacagactcacagtatgcattaggaatcattcaggcacaaccagacaggagtgaatcagagttagtcaatcaaataatagagaagctaataaaaaaggacaaagtctacctgtcatgggtaccagcacacaaagggattggaggaaatgaacaagtagataaattagtcagtaatggaatcaggaaggtactatttttagatggcatagataaagcccaagaagagcatgaaagatatcacagcaattggaaggcaatggctagtgattttaatctgccacctatagtagcaaaagaaatagtggccagctgtgataaatgtcagatgaaaggggaagccatgcatggacaagtagactgtggtccaggaatatggcaattagattgtacacatttagaagggaaaattatcttagtagcagtccatgtagccagtggctatatagaagcagaagttatcccagcagaaacaggacaggagacagcatactttatattaaaattagcaggaagatggccagtgaaagtaatacacacagacaatggcagcaatttcaccagtgctgcagtaaaggcggcatgttggtgggcaaatgtcacacaagaatttggaattccctacaatccccaaagccaaggagtagtggaatctatgaataaagaattaaagaaaattataggacaggtcagagatcaagctgaacaccttaagacagcagtacagatggcagtattcattcacaattttaaaagaaaaggggggattggggggtacagtgcaggggaaagaataatagatataatagcatcagatatacaaactaaagaactacaaaaacagattataaaaattcaaaattttcgggtttattacagggacagcagagaccccatttggaaaggaccagcaaaactactctggaaaggtgaaggggcagtagtaatacaggacaatagtgatataaaggtagtaccaagaagaaaagcaaaaatcattagggattatggaaaacagatggcaggtgatgattgtgtggcaggtagacaggatgaggattagaacatggaatagtttagtaaaacatcatatgtatgtctcaaagaaagctaagggttggttttatagacatcactttgaaagtaggcatccaaaagtatgttcagaagtacacatcccactaggggatgctaggttagtagtaagaacatattggggtctgcatacaggagaacgagactggcacttgggtcatggggtctccatagaatggaagcagaaaagatatagcacacaaatagaccctgacttagcagaccaactgattcacctgtattattttaattgtttttcagactctgccataagaaaagccatattaggagaaatagttagacctaggtgtgaatatcaagcaggacataataaggtaggatcgctacaatatttggcactgaatgctttagtagcaccaacaaagacaaagccacctttgcctagtgttaggaagttagcagaagacagatggaaagagccccagaagaccaggggccacagagggagccgtccaatgaatggacactagaactgttagaagagcttaaacatgaagctgttagacactttcctaggccgtggctccatggattaggacaacatatctatgaaacatatggggatacttgggaaggggttaaagctataataagaattttgcaacaactattgtttgttcatttcagaattgggtgtcaacatagcagaataggcattattcaagggagaagaggcaggaatggagctggtagatcctagcctagagccctggaaccacccgggaagtcagcctacaactgcttgtagcaagtgttactgtaaaatgtgctgctgcattgccaattgtgctttctgaacaagggcttaggcatctcctatggcaggaagaagcggagacgccgacgaggaactcctcagagccgtcaggatcatcaaaatcctgtaccaaagcagtgagtagtaataattagcatatgtgatgcaacctttaacaataactgcaatagtaggactagtagtagcattcatagcagccatagttgtgtggagcatagtatatatagaatataggaaaataaggaaacagaagaaaatagacaggttacttgatagaataagagaaagagcagaagatagtggcaatgagagtgatggggatacagaagaattatccactcttatggagatggggtacgaatatattttggataatgataatttgtaatgctgaacaattgtgggtcacggtctactatggggtacctgtgtggaagacggcagagaccaccctattttgtgcatcagatgctaaagcatatgatacagaagtacataatgtctgggctacacatgcctgtgtacccacagaccctaacccacaagaaatacatttggaaaatgtaacagaaaagtttaacatgtggaaaaataacatggtagaacagatgcatgaagatataattagtctatgggaccaaagcctaaagccatgtgtaaagttaacccctctctgcgttactttagattgtcataatttcaacaacagctacagcaacagtagcaacctaactagtgacatgaatggggaaataaaaaactgctctttcaatataaccacagaagtaagagataagaaaaagaagatgcatgcacttttttatagacttgatgtagtacaaattaatgaaaataatggtagtcagtataggttaataaattgtaatacctcagccattacacaggcttgtccaaaggtatcctttgagccaattcccatacattattgtgccccagctggttttgcgattctaaagtgtaaggataaagggttcaatggaacagggccatgcaaaaatgtcagcacagtacaatgcacacatggaatcaagccagtagtatcaactcaactgctgttaaatggcagtctagcagaaggagaagtagtgatcagatctgaaaatatcacaaacaatgccaaaaccataatagtacagttggctaatcctgtaaaaattaattgtaccagacctaacaacaatacaagaaaaggtgtacatataggaccagggcaagcattctatgcaacaggtgacataataggggatataagacaagcacattgtaatgtcagtaaaacagaatggaataaaactttacatcaggtagttacacaattaaagacgtactttaagaacaccacaataatctttgctaaccccttaggaggggatgtagaaattacaacacatagttttaattgtggaggagaatttttctattgcaatacatcaaaactgtttaatagcacttgggataatagcaatagcactgccaaccacacagggtcaaatgacactataactctccaatgcagaataaagcaaattgtaaatatgtggcagaaagtaggacaagcaatgtatgcccctcccatccaaggaataataagatgtgattcaaacattacaggactactattaacaagagatggagggaataatagtacaaatgaaaccttcaggcctggaggaggagatatgagggacaattggagaagtgaattatataagtataaagtagtaaaaattgaaccactaggtgtagcacccacccgtgcaaaaagaagagtggtggagagagaaaaaagagcggttggactgggagctgtcttccttgggttcttaggagcagcaggaagcactatgggcgcgcggtcaataacgctgacggtacaggccagacaattattgtccggcatagtgcaacagcagagcaatttgctgaaagctatagaggctcaacaacatctgttgaaactcacggtctggggcattaaacagctccaggcaagagtcctggctctggaaagatacctaagagatcaacagctcctaggaatttggggctgctctggaaaactcatctgcaccactactgtaccctggaactctagctggagtaataaaacttttaatgacatatgggataacatgacctggatacaatgggagaaagaaattagcaattacacagacataatatataatctaattgaagaatcgcagaaccggcaggaaaagaatgaacaagacttgttggcattggacaagtgggcaagtctatggaattggtttgacataacaaattggctatggtatataagaatatttataatgatagtaggaggtttaataggtttaagaatagtttttgctgtgcttactataataaatagagttaggcagggatactcacctttgtcgttccagacccttacccaccaccagagggaacccgacagacccgaaagaatcgaagaaggaggtggagagcaagacaaagacagatccgtgcgattagtgagcggattcttagcacttgcctgggacgatctgcggagcctgtgcctcttcagctaccaccgattgagagacttaatcttgattgcagcgaggactgtggaacttctgggacacaactgtctcaagggactgagactggggtggggagccctcaaatatctgtggaatcttatatcatactgggttcaggaactaaagaatagtgctattaatttgcttaatacaatagcaatagtagtagctaactggacagatagagctatagaaataggacaaagagtgggtagagctattcgcaacatacctagaagaatcagacagggcttcgaaagggctttgctataacatgggtggcaagtggtcaaaaagcagcatagtgggatggcctaaggttatgaaaagaatgagacaaacccctacagcagcaacaggagtaggagcagcatctcaagatttagatagacatggagcaatcacaagcagtaatacagcacaaactaaccctgattgtgcctggctggaagcacaagaggatgagaatgtaggctttccagtcaggccacaggtgcctttgagaccaatgacttataagggagctgtcgatctcagccactttttaaaagaaaaggggggactggatgggttaatttactccaagaaaagacaagaaatccttgatctgtgggtctataacacacaaggattcttcccagattggcagaactacacaccagggccagggactagattcccactgacctttgggtggtgcttcaaactagtaccaatggatccagcagaggtagaggaagccaatgaaggagagaacaacagtttattacaccccatctgtcaacatggaatggaggacgacgatagagaagtgctgatctggagatttgacagtagactggcattcagacacacagctagagagctgcatccggagtactacaaagactgctgacacagaagttgctgacagggactttccactggggactttccggggaggtgtggtttgggaggagttggggagtggctaaccctcagaccgtgcatataagcagctgcttctcgcctgtactgggtctctcttgctagaccagatctgagcctgggagctctctggctagcggaggaacccactgcttaagcctcaataaagcttgccttgagtgcttcaagtagtgtgtgcccgtctgattgtgtgactctggtaac

SEQ ID NO: 5 - DNA, 637 bp, Human immunodeficiency virus type 1
tgctgttaaatggcagtctagcagaagaaggagaaggtgtaatgattagatctgaaaatctcacaaacaatgccaaaaccataatagtacagcttggtacgcctgtaaacattagttgtaccagacccaacaacaatacaagaaaaagtgtacctataggaccaggacaagcattttatgcaacagagaacataataggggatataagacaagcatattgtaatgtcgatagaagaaaatggaatgaaattttacaaaaagtagctgaacaattaggacaacactttaacaaaacgataatttttactgaatcctcaggaggggatctagaaattacaacacatagctttaattgtggaggagaatttttctattgcaatacatcaggcctgtttaatagtacttggaatcagaatggcactgccatcacgcagaattcaacggataatggtactataactctctcatgtagaataaaacaaattgtacgtatgtggcagggagtaggacaagcaatgtatgcccctcccattcaaggagtaataaaatgtgtatcaaacattacaggactactgctagtaagagatggtgggaagagtaatagtacagaggagacgttcagacctggaggagggat

SEQ ID NO: 6 - DNA, 599 bp, Human immunodeficiency virus type 1
ttcactgccagtctagcagaaggaaagatagtgattagatctgaaaatatcacaaacaatgccaaaaccataatagtacacttagataagcctgtacaaatcagttgtaccagacccaccaataatactagaaaaggtggccatataggaccagggcaagcattctatgcaacaggtgccataacaggggatataagacaagcatattgtaatgtcagtagacaaaagtggaatgacactttaaaacaggtagctgaacaattaggggagtattttaagaataaaacaataatctttgctaacccctcaggaggggatttagaaattacaacacatagttttaattgtagaggagaatttttctattgcaatacatcaaaactgtttaatggcacttggagcaatgacactgacattataactctccaatgcagaataaagcaaattgtaaatatgtggcacaaagtaggacaagcaatgtatgcccctcccatcccaggaataataaggtgtgactcaaacattacaggactactattaacaagagatggaggacatagtaatagtacgaatgagactgtcaggcctggaggaggaga

SEQ ID NO: 7 - DNA, 551 bp, Human immunodeficiency virus type 1
aggtctgaaaacatcacagacaataccaaaaccataatagtgcagcttaataattctatagaaattaattgtaccagacccaacaacaatacaagaaaaagtataccattcggacctggacaagcattctatgcaacaggtgatataataggagatataagacaagcacattgtaatgtaagtagagaaaaatggaataagatgttacagaatgtaacagcacaactaaaaaaggtctttaataaagaaaatgtaacctttaaatcatctgcaggaggggacctagaaattacaacacatagttttaattgtagaggagaatttttctattgtaatacatcagaactgtttaatacatcaggattgcctaataataatactaatggcactatcacactcccatgtaagataaaacaaattataagaatgtggcagagagtgggacaagcaatgtatgcccctcccattgcaggagagattatatgtatatcaaacattacagggctaatattaacaagagatagtgtaggtaaggaaaataataatacctt